(19) United States
(12) Patent Application Publication
(10) Pub. No.: US 2026/0105998 A1
(43) Pub. Date: Apr. 16, 2026

(54) METHODS AND SYSTEMS FOR DESIGNING CLINICAL TRIALS

(71) Applicant: One2Treat, S.A., Mont-Saint-Guibert (BE)

(72) Inventors: Marc Eric Georges Raymond BUYSE, Genval (BE); Jean-Christophe CHIEM, Brussels (BE); Sébastien COPPE, Achêne (BE); Emilie BARRÉ, Arquennes (BE); Sarah KOSTA, Liège (BE); Samuel SALVAGGIO, Céroux-Mousty (BE); Benoît DAVID, Nil-Saint-Vincent (BE)

(21) Appl. No.: 19/354,392

(22) Filed: Oct. 9, 2025

Related U.S. Application Data
(60) Provisional application No. 63/705,860, filed on Oct. 10, 2024.

(51) Int. Cl. G16H 10/20; G16H 50/30

(52) U.S. Cl. CPC G16H 10/20; G16H 50/30

(57) ABSTRACT

In some aspects, the present disclosure provides methods and systems for evaluating treatment outcomes of a clinical intervention or for designing clinical trials. In some embodiments, the methods and systems described herein comprises receiving a user input, thereby generating prioritized treatment outcomes. Additionally, the methods and systems may involve using an artificial intelligence module.

### CROSS-REFERENCE

This application claims the benefit of U.S. Provisional Application No. 63/705,860, filed Oct. 10, 2024, which is incorporated herein by reference in its entirety.

### BACKGROUND

Proper design of clinical trials is crucial for optimizing resources, as these trials are costly and time-consuming. Statistical analysis of clinical data may be performed during the design process, ensuring that the trials are effectively structured and yield reliable results.

### SUMMARY

Conventional design of clinical trials focuses on a single primary outcome. Secondary outcomes, which are often affected by the clinical intervention studied in the trials, are usually not considered. This approach may result in suboptimal utilization of information and resources, leaving limited scope for incorporating patient voices and clinical insights. There exists a great need for better methods and systems for prioritizing treatment outcomes of clinical trials and for designing clinical trials.

Current approaches for determining clinical interventions in clinical trials face inefficiencies due to lack of patient-centric assessments. Statistical analysis of clinical data may be performed to identify treatment plans for subjects (e.g., patients in clinical trials) and to evaluate net benefits of treatment plans (e.g., net treatment benefits). Currently, there may be a lack of flexible approaches to identifying and evaluating net benefits of treatment plans (e.g., net treatment benefits). Thus, there exists a need for improved methods and systems which take into account personalized preferences and perform assessments at the patient-level or individual-level (e.g., using patient-level treatment outcomes and data).

The present disclosure provides methods for improving the efficiency and effectiveness of a clinical intervention. For example, during a clinical trial, the methods can allow a user to select a prioritization function that assigns ranked values to each of the plurality of treatment outcomes. The user can select the ranked values based on subject-level efficacies, subject-level adverse effects of individual treatment outcomes of the plurality of treatment outcomes on the subject, a personalized preference of the subject, or any combination thereof. The selection can then be used to generate net treatment benefits of clinical interventions and administer a clinical intervention as part of a clinical trial.

The present disclosure provides methods that can allow clinical trials to be performed with greater efficiency. For example, the claimed methods can generate a net benefit score representing the likelihood that a random patient in the experimental arm has a more favorable outcome in the trial than a random patient in the control arm. The net benefit value may be a holistic value (e.g., ranging from −1 to 1), which provides simple and intuitive means for a clinician or a patient to understand its significance. For instance. a value greater than 0 may signify that the experimental arm is preferred (based on the choice of priorities), while a value less than 0 may signify the opposite conclusion.

The present disclosure provides methods that can allow simulations of clinical trials to be performed in order to model conditions of a real clinical trial. The simulations can provide computer-simulated estimates and approximations of parameters of a clinical trial that would provide significant improvements to physical treatment benefits that patient-participants in the clinical trial obtain. By performing multiple simulations that consider the various possible outcomes of a clinical study based on the patient-participants' preferences, the simulations can provide computer-simulated estimate of the number of patient-participants that are likely necessary to obtain clinically validated results for the treatment under study. As a result, the method can increase treatment benefits to participants, reduce the number of patient-participants that do not obtain meaningful therapeutic benefits, and overall improve the speed efficiency, the cost efficiency, and the statistical certainty of a clinical trial.

In an aspect, the present disclosure provides a method for performing a clinical trial evaluating a clinical intervention for a plurality of subjects, comprising: a) obtaining a dataset for a treatment set of virtual subjects and a reference set of virtual subjects, wherein the treatment set of virtual subjects receives the clinical intervention and the reference set of virtual subjects does not receive the clinical intervention, and wherein the dataset comprises a plurality of treatment outcomes for the treatment set of virtual subjects and the reference set of virtual subjects; b) obtaining a prioritization function of the plurality of treatment outcomes; c) using the dataset and the prioritization function to perform a plurality of simulated clinical trials, thereby producing a set of simulated outcomes; d) processing the set of simulated outcomes to generate a set of simulated net treatment benefit scores and a set of simulated trial power scores, wherein the processing comprises performing a set of pairwise comparisons between a first subject selected from the treatment set of subjects and a second subject selected from the reference set of subjects for each of the simulated outcomes in the set of simulated outcomes; e) determining a number of subjects for the clinical trial based on the set of simulated net treatment benefit scores and the set of simulated trial power scores; f) selecting a treatment set of real subjects and a reference set of real subjects corresponding to the number of subjects; g) administering the clinical intervention to the treatment set of real subjects in the clinical trial; and h) determining an efficacy of the clinical intervention based on result of the clinical trial, wherein a net treatment benefit of the clinical trial is positive.

In an aspect, the present disclosure provides a method of using a prioritization function to assist in determination of a clinical intervention for a subject as part of a clinical trial, comprising: a) obtaining a dataset for a treatment set of subjects and a reference set of subjects, wherein the treatment set of subjects receives the clinical intervention during the clinical trial and the reference set of subjects does not receive the clinical intervention during the clinical trial, and wherein the dataset comprises a plurality of treatment outcomes for the treatment set of subjects and the reference set of subjects during the clinical trial; b) receiving, on a first computing device, the plurality of treatment outcomes for the treatment set of subjects and the reference set of subjects, wherein the plurality of treatment outcomes are transmitted over a computer network; c) presenting to a first user, via a first user interface on a first electronic display of the first computing device, a representation of the plurality of treatment outcomes; d) selecting, by the first user via the first user interface on the electronic display, a prioritization function that assigns ranked values to each of the plurality of treatment outcomes, wherein the ranked values are selected by the first user based at least in part on (1) subject-level efficacies and subject-level adverse effects of individual treatment outcomes of the plurality of treatment outcomes on the subject and (2) a personalized preference of the subject; e) performing, by a computer processor, a plurality of simulated clinical trials using the dataset and the prioritization function, thereby producing a set of simulated outcomes; f) processing the set of simulated outcomes to generate a set of simulated net treatment benefit scores and a set of simulated trial power scores, wherein the processing comprises performing a set of pairwise comparisons between a first subject selected from the treatment set of subjects and a second subject selected from the reference set of subjects for each of the simulated outcomes in the set of simulated outcomes; g) determining a parameter for the clinical trial based on the set of simulated net treatment benefit scores and the set of simulated trial power scores; and h) presenting to a second user, via a second user interface on a second electronic display of a second computing device, a report comprising the parameter for the clinical trial.

In an aspect, the present disclosure provides a computer program product comprising computer-executable instructions, comprising: a graphical user interface comprising a plurality of user-interactive elements, wherein the plurality of user-interactive elements are configured to receive a plurality of parameters for a clinical trial; a first set of computer-executable instructions to, upon receiving a user request via the graphical user interface: obtain a dataset for a treatment set of virtual subjects and a reference set of virtual subjects, wherein the treatment set of virtual subjects receives a clinical intervention during a virtual clinical trial and the reference set of virtual subjects does not receive the clinical intervention during a virtual clinical trial, and wherein the dataset comprises a plurality of treatment outcomes for the treatment set of subjects and the reference set of subjects during the clinical trial; perform a computer simulation of a plurality of simulated clinical trials to produce a set of virtual clinical trial outcomes using the dataset and a prioritization function; process the set of virtual clinical trial outcomes to generate a set of simulated net treatment benefit scores and a set of simulated trial power scores, by performing a set of pairwise comparisons between a first subject selected from the treatment set of virtual subjects and a second subject selected from the reference set of virtual subjects for each of the virtual clinical trial outcomes in the set of virtual clinical trial outcomes; output a recommended number of subjects for the clinical trial based on the set of simulated net treatment benefit scores and the set of simulated trial power scores; and display an electronic report comprising the recommended number of subjects for the clinical trial in the graphical user interface.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a table showing historical data and assumptions used to justify sample size calculation. (Abbreviations: ATO: arsenic trioxide; ATRA: all-trans retinoic acid; ChT: chemotherapy).

FIG. 2A illustrates power as a function of total sample size (equal allocation) for the test using the NTB with the assumptions shown in Table 1, based on 10,000 simulations. FIGS. 2B-2C illustrate power as a function of total sample size (equal allocation) for the test using the NTB with the assumptions described in Table 1, based on 10,000 simulations, assuming no dropouts. FIG. 2B uses the historical data from the CMC Vellore cohort to model dependencies across outcomes. FIG. 2C assumes complete independence between outcomes.

FIG. 3 depicts evolution of the NTB across successive prioritized outcomes. The black curve illustrates the initial ordering in the NTB. The other gray curves illustrate the NTB when the order of the toxicity outcomes is shuffled, leaving EFS as first priority. Notations: T=binary EFS outcome, Oj stands for “Outcome j,” j=1, . . . , 4, which may be Grade 3/4 documented infections, differentiation syndrome, hepatotoxicity, or neuropathy, depending on the order of priorities.

FIG. 4 is a table of results of a generalized pairwise comparison (GPC) analysis following the assumptions of the design in the case N=280.

FIG. 5 is a graphical illustration of the classification of pairs along the hierarchical procedure underlying the NTB. Each level of the prioritized outcomes represents the proportion of pairs classified as either favorable, unfavorable, or ties on that particular outcome, in addition to the numerical contribution of the outcome to the overall NTB. Pairs that are classified as ties on one prioritized outcome are evaluated on the next level of the hierarchy of outcomes.

FIG. 6 depicts a tipping-point analysis, showing values of the NTB as a function on assumptions on EFS in both arms, keeping every other aspect of the design fixed. The order of priorities is the one chosen for the design. The NTB is positive (favoring the reduced dose) for situations depicted in green, negative (favoring the standard dose) for situations depicted in red, and zero (favoring neither treatment) for situations depicted in orange. The value of the actual design is highlighted in the hexagon.

FIG. 7 illustrates the ergonomic design of the methods, which allows intuitive drawing of the correlation of various clinical outcomes. Rapid modification on a visual representation can support a discussion between the experts to reach a consensus or consider several scenarios of dependency.

FIG. 8 shows an example of a computer system.

FIG. 9 illustrates user input elicitation. Exemplary treatment outcomes (e.g., survival, toxicity and quality of life) are shown in a pair of clinical scenarios. The user may compare the pairwise clinical scenarios and prioritize the treatment outcomes based on their preference.

FIG. 10 illustrates user input elicitation with graphical user interface (GUI).

FIG. 11 illustrates user input elicitation. Exemplary treatment outcomes (e.g., survival, toxicity and quality of life) are shown in a pair of clinical scenarios. The user may compare the pairwise clinical scenarios and prioritize the treatment outcomes based on their preference.

FIG. 12 illustrates a basic information GUI. The GUI can display criteria for treatment outcomes, e.g., crisis duration, crisis frequency, and/or crisis intensity in the case of a treatment against migraine. The treatment outcomes can be continuous or categorized. For example, crisis duration can be categorized into 1, 2, 6, or 12 hours; crisis frequency can be categorized into daily, weekly, monthly, or yearly; crisis intensity can be categorized into low, medium, or high. The GUI can display attributes of respondents (clinicians, patients, stakeholders). For example, respondents attributes can comprise age group, ethnicity, gender, and/or specialty.

FIG. 13 illustrates an analytics GUI. The GUI can display analytics for survey data. The GUI can display the number of surveys reported, the date and/or the time at which surveys are reported, survey durations, and/or statistics thereof.

FIG. 14 illustrates a priority distribution GUI. The GUI can display a distribution of survey responses that select particular treatment outcomes. For example, the GUI can display a distribution of survey responses that selected crisis duration, crisis frequency, and/or crisis intensity as a first, second, or third priority.

FIG. 15 illustrates a priority flow GUI. The GUI can display a diagram that shows how survey respondents prioritized different treatment outcomes. For example, the diagram can show what number, fraction, or percentage of survey respondents prioritized which treatment outcome as first, second, third, . . . and so forth.

FIG. 16 illustrates a threshold distribution GUI. The GUI can display for each treatment what number, fraction, or percentage of survey respondents provided responses which generated a specific threshold value.

FIG. 17 illustrates a score analysis GUI. The GUI can display scores of agreements measuring the ability of a priority function to reproduce the classification of a survey and their parameters. FA and TA are measures of agreement, i.e., the capacity to classify the same pairs (TA) and with the same driving outcome (FA)

FIG. 18 illustrates a priority funnel GUI. The GUI can display the selected priority function. For example, this solution can rank 1) Intensity with threshold=1 over, 2) Frequency with threshold 86%, over 3) Duration with threshold 4%. This graphs also shows the capacity of this priority function to classify the pairs of other surveys (in average 60%).

FIG. 19 illustrates a classification flow GUI. For the selected priority function, the GUI can display the number, fraction, or percentage of surveys that reproduce the correct results. For example, 97% of survey respondents can be captured by providing predicted treatment outcomes with a crisis intensity of at most threshold=1, frequency with a threshold of 86%, and a duration with a threshold of 4%.

### DETAILED DESCRIPTION

As used herein, the singular form “a”, “an”, and “the” include plural references unless the context clearly dictates otherwise. For example, the term “an intervention” includes a plurality of interventions.

As used herein, the term “subject,” generally refers to an entity or a medium that has testable or detectable genetic information. A subject may be a person, individual, or patient. A subject may be a vertebrate, such as, for example, a mammal. Non-limiting examples of mammals include humans, simians, farm animals, sport animals, rodents, and pets. The subject may be displaying a symptom(s) indicative of a health or physiological state or condition of the subject. As an alternative, the subject may be asymptomatic with respect to such health or physiological state or condition. The subject may be an adult or a child.

### Designing Clinical Trials

In many clinical settings, particularly in randomized trials of experimental therapies, multiple outcomes, or endpoints, are of interest. The conventional design of clinical trials focuses on a primary outcome that is both clinically relevant and statistically sensitive enough to detect differences between an experimental treatment and standard care. However, during the design stages, secondary outcomes, which may be affected by the clinical intervention, are often not considered. This approach can result in suboptimal utilization of information and leaves limited scope for incorporating patient voices and clinical insights, which may highlight the multifaceted impacts of treatments on aspects like safety, efficacy, quality of life, and patient-reported outcomes.

The present disclosure provides improved methods and systems for designing clinical trials by including Generalized Pairwise Comparison (GPC), which enables the clinical trials with the Net Treatment Benefit (NTB) as a primary outcome. By adopting GPC in the design of clinical trials, researchers can shift from a traditional single-outcome focus to an approach that integrates the patients' preferences regarding the outcomes affected by the clinical interventions. This multidimensional approach to trial design more accurately reflects the varied objectives pursued by a trial. It may also lead to reductions in required sample sizes depending on the context. The methods and systems for designing clinical trials includes conducting statistical analysis on historical clinical data, which may allow the consideration of a plurality of prioritized treatment outcomes. Inputs from a user, such as a medical professional or a patient, may be incorporated into the statistical analysis, so that statistical inferences over the underlying clinical data may be made with granularity. Users may provide their input by comparing a plurality of pairwise clinical scenarios, thereby generating their prioritized treatment outcomes for broader applications in designing clinical trials. The pairwise clinical scenarios can be provided on a graphical user interface (see, e.g., FIG. 10) based on history clinical cases. In some embodiments, the pairwise scenarios are provided by artificial intelligence. The methods and systems provided herein may be applied to designing many aspects of a clinical trial using the NTB as a primary endpoint, for example, calculating the number of human subjects (e.g., sample size) required for the trial. There is a particular challenge of modeling multiple endpoints in a trial as the mutual correlation and dependency structure of the clinical outcomes are required for the computation. The methods and systems provided herein address this challenge by retrieving patterns from historical data and/or receiving user input via elicitation, thereby acquiring information on the mutual correlation and dependency structure of the clinical outcomes required for modeling multiple endpoints of a clinical trial.

In some aspects, the present disclosure provides a computer-implemented method for designing a trial evaluating a clinical intervention in a subject, comprising: obtaining a dataset for a treatment set of subjects and a reference set of subjects, wherein the treatment set of subjects receives the clinical intervention and the reference set of subjects does not receive the clinical intervention, and wherein the dataset comprises a plurality of treatment outcomes for the treatment set of subjects and the reference set of subjects; receiving user input of a prioritization function of the plurality of treatment outcomes; generating a plurality of simulated trials, at least in part by: i) performing a set of pairwise comparisons between a first subject selected from the treatment set of subjects and a second subject selected from the reference set of subjects, based at least in part on the prioritization function, and ii) determining a net treatment benefit of the clinical intervention, based at least in part on the set of pairwise comparisons; and determining a parameter of the trial based at least in part on the plurality of simulated trials. The trial evaluating a clinical intervention can be referred to as an interventional clinical trial, an experimental clinical trial, or a clinical trial.

In another aspect, the present disclosure provides a computer-implemented method for designing a trial evaluating a clinical intervention in a subject, comprising: a) obtaining a dataset for a treatment set of subjects and a reference set of subjects, wherein the treatment set of subjects receives the clinical intervention and the reference set of subjects does not receive the clinical intervention, and wherein the dataset comprises a plurality of treatment outcomes for the treatment set of subjects and the reference set of subjects, b) obtaining a prioritization function of the plurality of treatment outcomes; c) using the dataset and the prioritization function to perform a plurality of simulated trials, thereby producing a set of simulated outcomes; d) processing the set of simulated outcomes, wherein the processing comprises performing a set of pairwise comparisons between a first subject selected from the treatment set of subjects and a second subject selected from the reference set of subjects; and e) determining a parameter of the trial based at least in part on the plurality of simulated trials.

Designing a clinical trial involves careful planning and consideration of a plurality of trial parameters. In some embodiments, the method of designing a trial provided herein comprises determining one or more parameters of the clinical trial based at least in part on the plurality of simulated trials, wherein the one or more parameters comprise a member selected from the group consisting of an objective, type of study design, study group, study population, sample size, intervention, endpoint, outcome, randomization, blinding, data collection, data cleaning, data storage, statistical test, power, significance level, missing data, and dropout rate. In some embodiments, the one or more parameters of the trial comprises an objective, type of study design, study group, study population, sample size, intervention, endpoint, outcome, randomization, blinding, data collection, data cleaning, data storage, statistical test, power, significance level, missing data, dropout rate, or any combination thereof. In some embodiments, the method provided herein comprises determining an endpoint, sample size, or power. In some embodiments, the method provided herein comprises determining an endpoint. In some embodiments, the endpoint comprises a plurality of ranked or prioritized endpoints. In some embodiments, the method provided herein comprises determining a sample size. In some embodiments, the method provided herein comprises determining a power. In some embodiments, the method provided herein comprises determining a function of sample size and power (e.g., g., a power function), based at least in part on the plurality of simulated trials.

In some embodiments, the power of the trial is a function of the sample size. In some embodiments, the sample size of the trial is at most about 500, at most about 450, at most about 400, at most about 350, at most about 300, at most about 250, at most about 200, at most about 150, at most about 100, or at most about 50 subjects. In some embodiments, the sample size of the trial is at least about 500, at least about 450, at least about 400, at least about 350, at least about 300, at least about 250, at least about 200, at least about 150, at least about 100, or at least about 50 subjects.

In some embodiments, the type of study design comprises a randomized controlled trial (RCT). The RCT may have a cross-over design or a parallel design. The RCT with a parallel design may comprise a non-inferiority (NI) trial, an equivalence trial, or a superiority trial. In some embodiments, the trial provided herein comprises an NI trial. In some embodiments, the clinical trial provided herein comprises a case group, a treatment group, a control group, a subgroup, or any combinations thereof. In some embodiments, the clinical trial compares the clinical intervention provided herein to a standard of care treatment. In some embodiments, the clinical intervention comprises an intervention that may be compared between a case group and a control group. In some embodiments, the clinical intervention comprises an intervention that may be compared between one case group and another case group. In some embodiments, the clinical intervention comprises an intervention that may be compared within a case group at various timepoints of the treatment.

In some embodiments, the method provided herein comprises generating at least 100, at least 200, at least 300, at least 400, at least 500, at least 600, at least 700, at least 800, at least 900, at least 1000, at least 1500, at least 2000, at least 2500, at least 3000, at least 3500, at least 4000, at least 4500, at least 5000, at least 5500, at least 6000, at least 6500, at least 7000, at least 7500, at least 8000, at least 8500, at least 9000, at least 9500, or at least 10000 simulated trials. In some embodiments, the method comprises generating a plurality of simulated trials with a plurality of sample sizes. In some embodiments, the sample size of the simulated trial is at least about 500, at least about 450, at least about 400, at least about 350, at least about 300, at least about 250, at least about 200, at least about 150, at least about 100, or at least about 50 subjects.

In some embodiments, the method of designing a trial further comprises modeling a set of variables of the plurality of simulated trials. In some embodiments, modeling the set of variables is based on historical data or based on user input. In some embodiments, user input comprises the user input elicited from experts, as described herein.

In some embodiments, the variables of the plurality of simulated trials comprise a sample size, endpoint, randomization, power, dropout rate, dependency of treatment outcomes, variance of treatment outcomes, or minimal detectable treatment outcome. In some embodiments, the variables of the plurality of simulated trials comprise a dropout rate of the plurality of simulated trials, a dependency of treatment outcomes, a variance of treatment outcomes, or a minimal detectable treatment outcome. In some embodiments, the method comprises modeling the dependency of treatment outcomes of the plurality of simulated trials. In some embodiments, the method further comprises receiving user input on the dependency of treatment outcomes. In some embodiments, the method further comprises modeling the dependency of treatment outcomes based on historical data.

### Simulating Clinical Trials

In an aspect, the present disclosure provides a method for performing a clinical trial evaluating a clinical intervention for a plurality of subjects. In some embodiments, the method can comprise obtaining a dataset for a treatment set of virtual subjects. In some embodiments, the method can comprise obtaining a dataset for a reference set of virtual subjects. In some embodiments, the treatment set of virtual subjects receives the clinical intervention. In some embodiments, the reference set of virtual subjects does not receive the clinical intervention. In some embodiments, the dataset comprises a plurality of treatment outcomes for the treatment set of virtual subjects. In some embodiments, the dataset comprises a plurality of treatment outcomes for the reference set of virtual subjects.

In some embodiments, the method comprises obtaining a prioritization function of the plurality of treatment outcomes. In some embodiments, the prioritization function comprises at least one of an ordering, a ranking, a set of weights, and a non-transitive ordering for individual treatment outcomes of the plurality of treatment outcomes. In some embodiments, the set of weights comprises non-zero weights. In some embodiments, the set of weights comprises zero weights. In some embodiments, weights are expressed as a value between 0 and 1. In some embodiments, weights are expressed in percentages (e.g., with the sum of weights adding to 1). In some embodiments, a non-transitive ordering may refer to an ordering with binary relations that are not transitive relations. In some embodiments, a non-transitive ordering may be synthesized from the plurality of treatment outcomes. In some embodiments, a non-transitive ordering may be synthesized from the plurality of treatment outcomes and a prioritization order or ranking of the plurality of treatment outcomes.

In some embodiments, the method comprises using the dataset and the prioritization function to perform a plurality of simulated clinical trials, thereby producing a set of simulated outcomes. In some embodiments, the plurality of simulated clinical trials are performed by a set of pairwise comparisons between a first subject selected from the treatment set of virtual subjects and a second subject selected from the reference set of virtual subjects, based at least in part on the prioritization function. In some embodiments, the method comprises processing the set of simulated outcomes to generate a set of simulated net treatment benefit scores and a set of simulated trial power scores.

In some embodiments, the method comprises determining a number of subjects for the clinical trial based on the set of simulated net treatment benefit scores and the set of simulated trial power scores. In some embodiments, the number of subjects for the clinical trial can be less than 10%, 20%, 30%, 40%, 50%, 60%, 70%, 80%, or 90% of a human estimate for the number of subjects for the clinical trial that does not account for the prioritization function or the plurality of treatment outcomes.

In some embodiments, the method comprises selecting a treatment set of real subjects and a reference set of real subjects corresponding to the number of subjects. In some embodiments, the treatment set of real subjects and the reference set of real subjects can be selected based on the real subjects' reported treatment outcome priorities.

In some embodiments, the method comprises administering the clinical intervention to the treatment set of real subjects in the clinical trial. In some embodiments, the method comprises determining an efficacy of the clinical intervention based on result of the clinical trial, wherein a net treatment benefit of the clinical trial is positive. In some embodiments, the net treatment benefit of the clinical trial is zero or positive for all of the treatment set of real subjects. In some embodiments, the net treatment benefit of the clinical trial as a whole is positive.

### Computer Method and System for Clinical Trial Simulations

In an aspect, the present disclosure provides a method of using a graphical user interface to assist in determination of a clinical study parameters. In some embodiments, the method comprises obtaining a dataset for a treatment set of subjects. In some embodiments, the method comprises obtaining a dataset for a reference set of subjects. In some embodiments, the treatment set of subjects receives the clinical intervention during the clinical trial. In some embodiments, the reference set of subjects does not receive the clinical intervention during the clinical trial. In some embodiments, the dataset comprises a plurality of treatment outcomes for the treatment set of subjects and the reference set of subjects during the clinical trial.

In some embodiments, the method comprises receiving, on a first computing device, the plurality of treatment outcomes for the treatment set of subjects and the reference set of subjects. In some embodiments, the plurality of treatment outcomes are transmitted over a computer network. In some embodiments, the method comprises presenting to a first user, via a first user interface on a first electronic display of the first computing device, a representation of the plurality of treatment outcomes.

In some embodiments, the method comprises selecting, by the first user via the first user interface on the electronic display, a prioritization function that assigns ranked values to each of the plurality of treatment outcomes. In some embodiments, the ranked values are selected by the first user based at least in part on subject-level efficacies. In some embodiments, the ranked values are selected by the first user based at least in part on subject-level adverse effects of individual treatment outcomes of the plurality of treatment outcomes on the subject. In some embodiments, the ranked values are selected based on a personalized preference of the subject.

In some embodiments, the method comprises performing, by a computer processor, a plurality of simulated clinical trials using the dataset and the prioritization function, thereby producing a set of simulated outcomes.

In some embodiments, the method comprises processing the set of simulated outcomes to generate a set of simulated net treatment benefit scores and a set of simulated trial power scores. In some embodiments, the processing comprises performing a set of pairwise comparisons between a first subject selected from the treatment set of subjects and a second subject selected from the reference set of subjects for each of the simulated outcomes in the set of simulated outcomes. In some embodiments, the method comprises determining a parameter for the clinical trial based on the set of simulated net treatment benefit scores and the set of simulated trial power scores. In some embodiments, the method comprises presenting to a second user, via a second user interface on a second electronic display of a second computing device, a report comprising the parameter for the clinical trial.

In an aspect, the present disclosure provides a computer program product comprising computer-executable instructions. In some embodiments, the computer-executable instructions comprises a graphical user interface comprising a plurality of user-interactive elements. In some embodiments, the plurality of user-interactive elements are configured to receive a plurality of parameters for a clinical trial.

In some embodiments, the computer-executable instructions comprises a first set of computer-executable instructions. In some embodiments, the computer-executable instructions are configured to be executed upon receiving a user request via the graphical user interface. In some embodiments, the computer-executable instructions are configured to obtain a dataset for a treatment set of virtual subjects and a reference set of virtual subjects. In some embodiments, the treatment set of virtual subjects receives a clinical intervention during a virtual clinical trial and the reference set of virtual subjects does not receive the clinical intervention during a virtual clinical trial. In some embodiments, the dataset comprises a plurality of treatment outcomes for the treatment set of subjects and the reference set of subjects during the clinical trial.

In some embodiments, the computer-executable instructions are configured to perform a computer simulation of a plurality of simulated clinical trials to produce a set of virtual clinical trial outcomes using the dataset and a prioritization function.

In some embodiments, the computer-executable instructions are configured to process the set of virtual clinical trial outcomes to generate a set of simulated net treatment benefit scores and a set of simulated trial power scores. In some embodiments, the computer-executable instructions are configured to perform a set of pairwise comparisons between a first subject selected from the treatment set of virtual subjects and a second subject selected from the reference set of virtual subjects for each of the virtual clinical trial outcomes in the set of virtual clinical trial outcomes. In some embodiments, the computer-executable instructions are configured to output a recommended number of subjects for the clinical trial based on the set of simulated net treatment benefit scores and the set of simulated trial power scores. In some embodiments, the computer-executable instructions are configured to display an electronic report comprising the recommended number of subjects for the clinical trial in the graphical user interface.

### User Input Elicitation

A clinical intervention may result in a plurality of treatment outcomes. In various clinical scenarios, a user may prefer certain treatment outcomes of a clinical intervention over others. In some embodiments, a user can comprise a medical professional, a patient, a caretaker of the patient, or a stakeholder of a trial). Users may prefer or prioritize certain treatment outcomes based on many factors, such as values, experiences, and treatment goals. In some cases, the user may prefer treatments supported by strong clinical evidence and prioritize efficacy. In other cases, the user may prioritize minimizing adverse effects and ensuring long-term safety. In addition to safety and efficacy, users may consider other factors when prioritizing treatment outcomes, including, but not limited to, cost-effectiveness, ease of administration, and overall quality of life. It is important to consider the user input when evaluating treatment outcomes of a clinical intervention or designing a clinical trial.

In some embodiments, the user input provided herein is used to generate the prioritization function for treatment outcomes described herein. In some embodiments, the user input provided herein is used to determine the dependency of treatment outcomes. In some embodiments, the user input provided herein is used to determine the threshold of clinical relevance.

In some aspects, the present disclosure provides improved methods and systems for evaluating a plurality of treatment outcomes of one or more clinical interventions, at least in part by receiving user input on clinical scenarios. In some embodiments, provided herein comprises a computer-implemented method for evaluating treatment outcomes of a clinical intervention in a subject, comprising: a) obtaining a dataset for a treatment set of subjects and a reference set of subjects, wherein the treatment set of subjects receives the clinical intervention and the reference set of subjects does not receive the clinical intervention, and wherein the dataset comprises a plurality of treatment outcomes for the treatment set of subjects and the reference set of subjects; b) receiving user input on a set of pairwise clinical scenarios, and c) generating a prioritization function of the plurality of treatment outcomes, based at least in part on the user input.

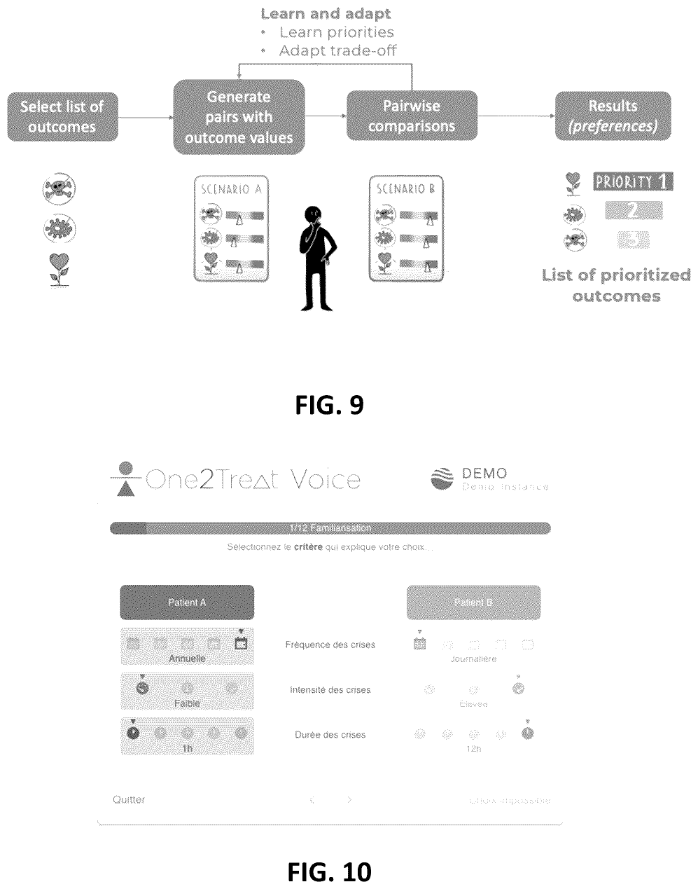

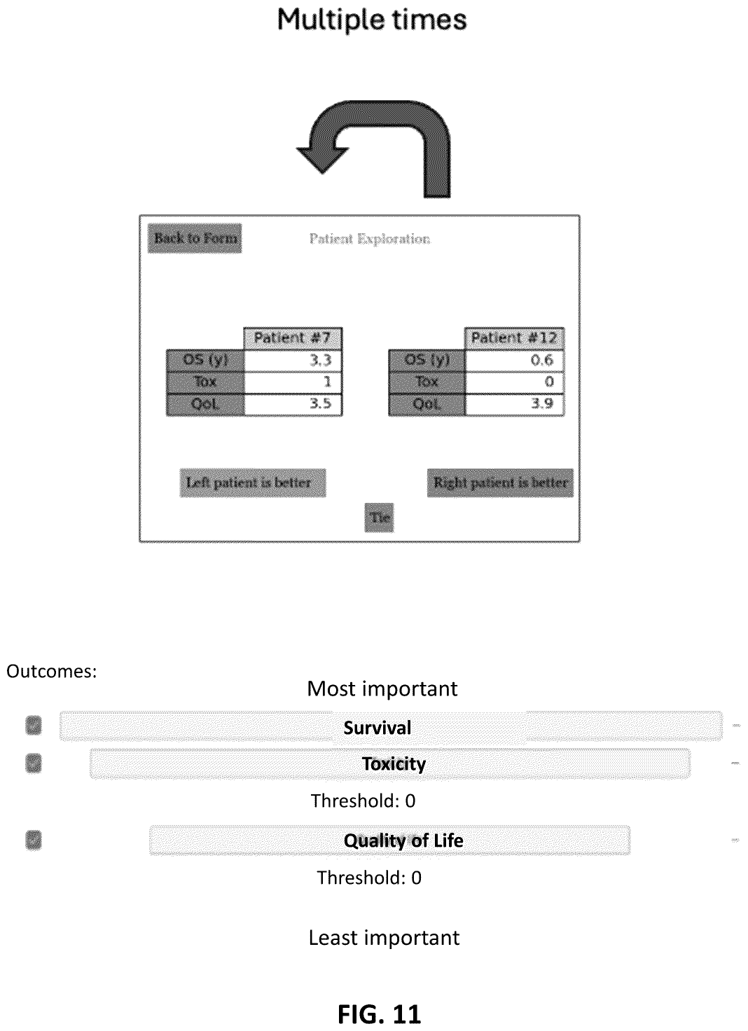

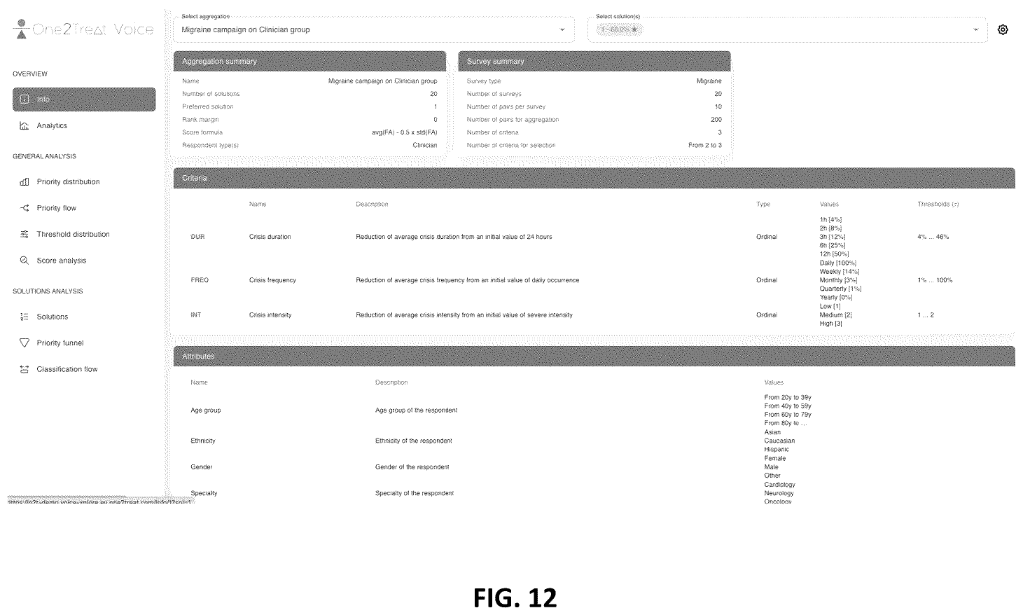

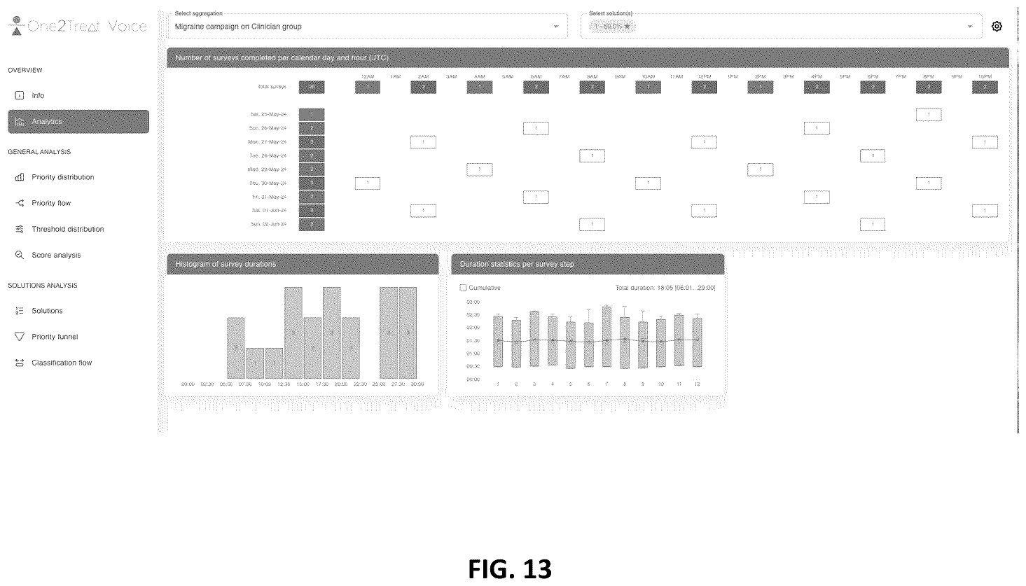

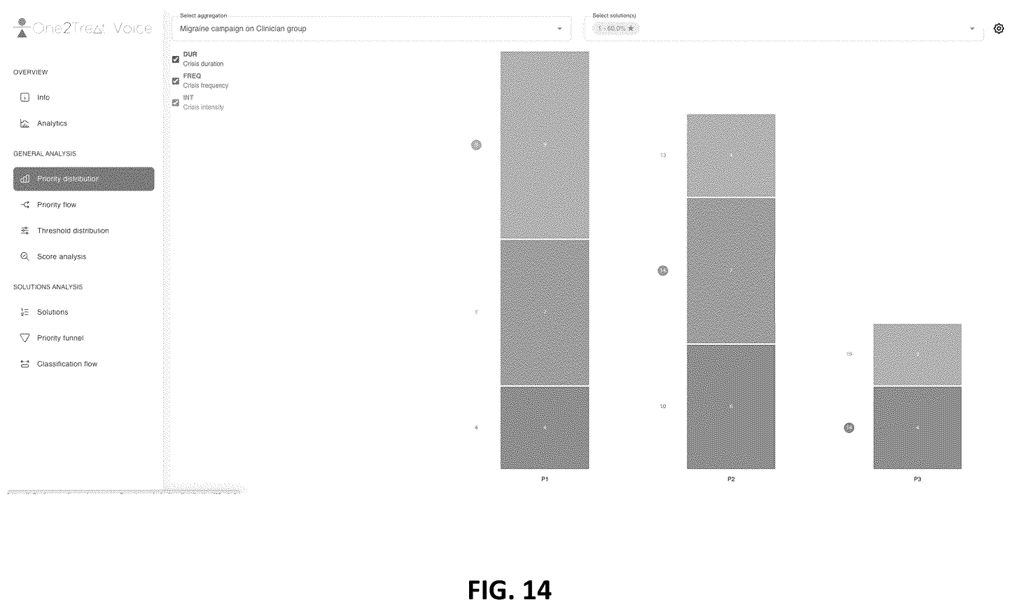

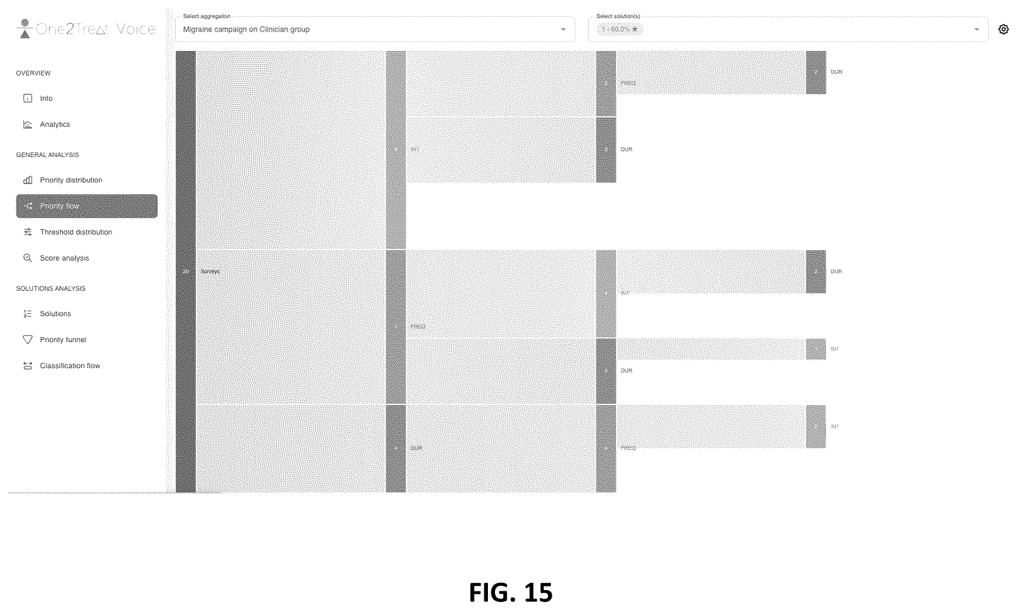

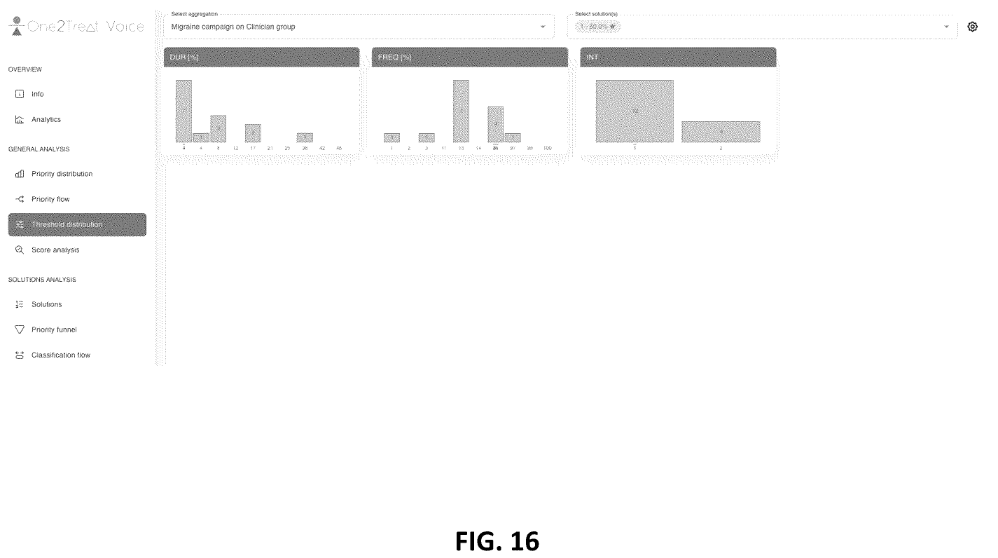

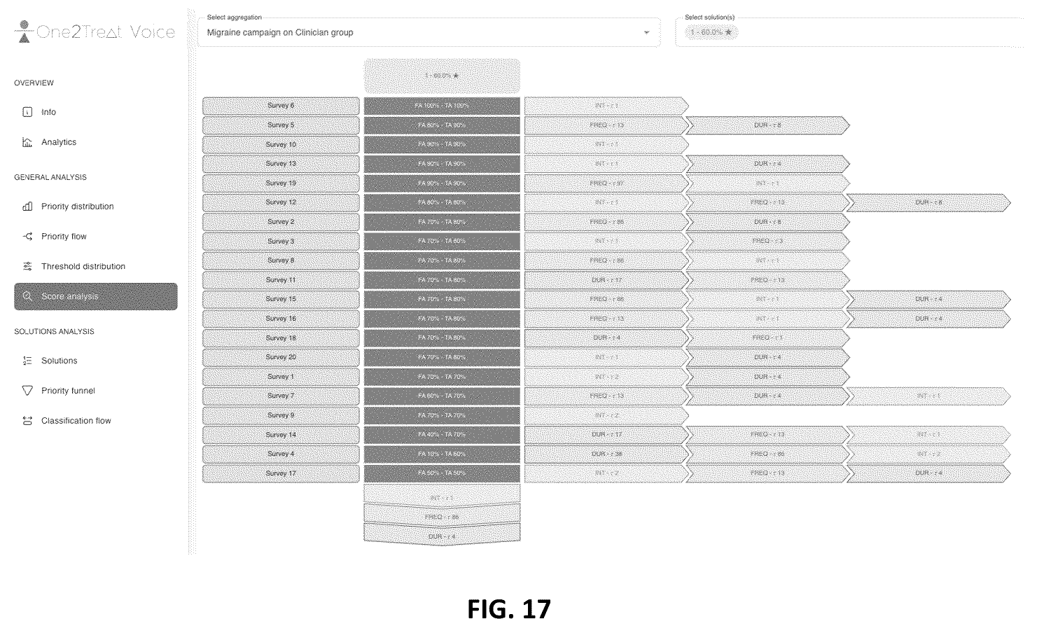

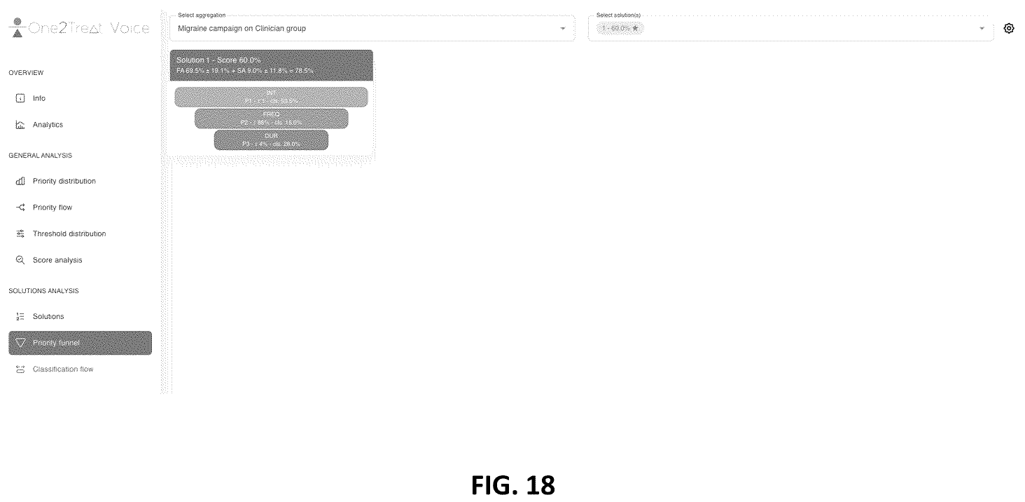

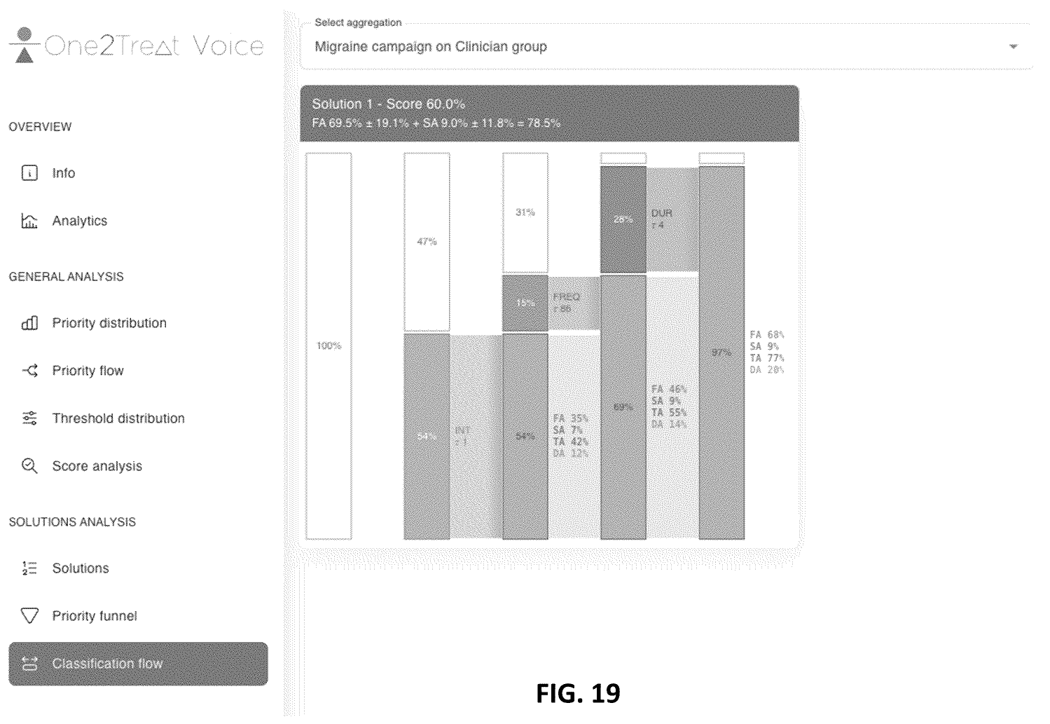

In some embodiments, the clinical scenarios are presented in a pairwise fashion, as one or more sets of pairwise clinical scenarios. In some embodiments, the set of pairwise clinical scenarios comprises at least 1 pair, at least 5 pairs, at least 10 pairs, at least 15 pairs, at least 20 pairs, at least 25 pairs, at least 30 pairs, at least 35 pairs, at least 40 pairs, at least 45 pairs, at least 50 pairs, at least 55 pairs, at least 60 pairs, at least 65 pairs, at least 70 pairs, at least 75 pairs, at least 80 pairs, at least 85 pairs, at least 90 pairs, at least 95 pairs, or at least 100 pairs of clinical scenarios. In some embodiments, the set of pairwise clinical scenarios comprises at most 1 pair, at most 5 pairs, at most 10 pairs, at most 15 pairs, at most 20 pairs, at most 25 pairs, at most 30 pairs, at most 35 pairs, at most 40 pairs, at most 45 pairs, at most 50 pairs, at most 55 pairs, at most 60 pairs, at most 65 pairs, at most 70 pairs, at most 75 pairs, at most 80 pairs, at most 85 pairs, at most 90 pairs, at most 95 pairs, or at most 100 pairs of clinical scenarios. In some embodiments, the set of pairwise clinical scenarios comprises at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 sets of pairwise clinical scenarios, e.g., as depicted in FIG. 9. In some embodiments, the set of pairwise clinical scenarios comprises at most 1, at most 5, at most 10, at most 15, at most 20, at most 25, at most 30, at most 35, at most 40, at most 45, at most 50, at most 55, at most 60, at most 65, at most 70, at most 75, at most 80, at most 85, at most 90, at most 95, or at most 100 sets of pairwise clinical scenarios, e.g., as depicted in FIG. 9.

In some embodiments, the pairwise clinical scenarios comprise clinical vignettes (e.g., patient treatment cases) with treatment outcomes of a clinical intervention. In some embodiments, the pairwise clinical scenarios can be presented to the user on a graphical interface, for example, as depicted in FIG. 10. The user may compare the pairwise clinical scenarios and prioritize the treatment outcomes based on their preference. In some embodiments, the pairwise clinical scenarios can be presented to the user in an online survey. The user may compare a preferred scenario from the pair of clinical scenarios. In some embodiments, user input from many users can be aggregated. In some embodiments, priorities, weights or thresholds of a plurality of treatment outcomes may be established based on the user input.

In some embodiments, user input can be elicited from at least 1, at least 2, at least 3, at least 4, at least 5, at least 6, at least 7, at least 8, at least 9, at least 10, at least 15, or at least 20 users. In some embodiments, user input can be elicited from at least 10, at least 20, at least 30, at least 40, at least 50, at least 60, at least 70, at least 80, at least 90, at least 100, at least 200, at least 300, at least 400, or at least 500 users. In some embodiments, user input can be elicited from at least 1, at least 2, at least 3, at least 4, at least 5, at least 6, at least 7, at least 8, at least 9, at least 10, at least 15, or at least 20 users. In some embodiments, user input can be elicited from at most 10, at most 20, at most 30, at most 40, at most 50, at most 60, at most 70, at most 80, at most 90, at most 100, at most 200, at most 300, at most 400, or at most 500 users.

In some embodiments, the methods and systems provided herein includes using Artificial Intelligence (AI). In some embodiments, the clinical vignettes described herein are obtained from a database of clinical cases. In some embodiments, AI may retrieve or create the clinical vignettes that are representative of real-life patient cases. In some embodiments, AI may retrieve or create clinical scenarios at least in part based on the empirical preferences of the user. In some embodiments, AI may comprise a large language models (LLM) module. In some embodiments, the LLM module may provide interactions to the user. For example, the LLM module may explain the clinical scenarios or statistical results, or generate automated reports.

### Interventions and Treatment Outcomes

In some embodiments, the clinical intervention comprises a pharmacological treatment (e.g., a drug treatment) or a non-pharmacological treatment. Pharmacological treatments may comprise a small molecule drug or a biologic drug. In some embodiments, the biologic drug comprises a peptide, a protein, an antibody or an antigen binding fragment thereof, nucleic acids, a cell-based therapy, or any combination thereof. The drug treatment may comprise medications to be administered via a plurality of routes. In some embodiments, the drug treatment is administered orally, topically, or by injections. Topical administration may comprise intranasal administration, intravaginal administration, transdermal administration, or inhalation. Injections may comprise intravenous, subcutaneous, intramuscular, or intrathecal administration. In some embodiments, non-pharmacological treatment comprises a medical device (e.g., laser or photodynamic therapy, radiofrequency or thermal ablation, or cryotherapy), a surgical intervention (e.g., invasive, minimally invasive, laparoscopic, or others), radiotherapy, radioisotopic/nuclear therapy, physical therapy, occupational therapy, phonoaudiological therapy, a rehabilitation intervention, a digital health intervention, a behavioral intervention, a psychological intervention, or any combination thereof.

In some embodiments, the clinical intervention comprises an approved treatment. In some embodiments, the clinical intervention comprises an experimental treatment. In some embodiments, the clinical intervention comprises an approved drug treatment. The clinical intervention may comprise administering the approved drug to trial subjects at a dosing regimen that is different from the approved dose. The clinical intervention may comprise administering the approved drug to trial subjects who have a disease or disorder that is not the approved indication of the approved drug. In some embodiments, the drug treatment comprises an experimental drug treatment. In some embodiments, the clinical intervention comprises a treatment for a cancer, an infectious disease, a cardiovascular disease, a central nervous system (CNS) disorder, an immunological disorder, or a metabolic disease. In some embodiments, the clinical intervention comprises a treatment for a cancer. In some embodiments, the clinical intervention of the trial is compared to a standard of care therapy.

Various forms of data may be used to measure treatment outcomes. In some embodiments, treatment outcomes are measured by discrete variables or continuous variables. In some embodiments, treatment outcomes are measured by categorial variables, true or false variables, color variables, or any combination thereof. Any number of variables may be used to measure a treatment outcome. In some embodiments, a treatment outcome is measured by 1 variable, 2 variables, 3 variables, 4 variables, 5 variables, 6 variables, 7 variables, 8 variables, 9 variables, 10 variables, or more than 10 variables.

The plurality of treatment outcomes may include various kinds of treatment outcomes. In some embodiments, the treatment outcomes comprise event-free survival time, progression-free survival time, overall survival time, another time to an event, efficacy, safety, quality of life, cost-related information, a biomarker, or any combination thereof. In some embodiments, the treatment outcomes comprise a score or a grade, wherein the score or grade may comprise a functional score, a performance score, a toxicity grade, a cancer grade, a grade in symptoms, a pain score, a behavioral score, a composite score, an index score, or any combination thereof. In some embodiments, the treatment outcomes comprise a member selected from the group consisting of event-free survival time, progression-free survival time, overall survival time, another time to an event, efficacy, safety, quality of life, cost-related information, and a biomarker.

In some embodiments, event-free survival time may refer to the duration of time between an end of a treatment and future occurrence of a medical event related to the treatment or the condition the treatment is designed to treat. In some embodiments, event-free survival time may refer to the duration of time between an end of a treatment and death related to the treatment or the condition the treatment is designed to treat. In some embodiments, event-free survival time may refer to the duration of time between an end of a treatment and death related to natural causes. In some embodiments, progression-free survival time may refer to the duration of time between an end of a treatment and a progression in the condition that the treatment is designed to treat. In some embodiments, progression-free survival time may refer to the duration of time between an end of a treatment and a progression in the condition that the treatment is designed to treat. In some embodiments, a progression-free survival time may refer to the duration of time between an end of a treatment and death related to natural causes.

In some embodiments, efficacy may refer to the efficacy of a clinical intervention or a treatment in providing a therapeutic benefit. In some embodiments, a therapeutic benefit comprises an increase in self-perceived well-being, an increase in well-being observed by a medical professional, an increase in self-perceived happiness, an increase in happiness observed by a medical professional, an increase in self-perceived mental health, an increase in mental health observed by a medical professional, an increase in physical activity, an increase in mental activity, an increase in sleep quality, an increase in metabolism, an increase in brain activity, an increase in hearing ability, an increase in sense of smell, an increase in visual acuity, an increase in the ability to taste, an increase cell regeneration, an increase in immune system strength, an increase in blood flow, an increased appetite, a reduction in pain, a reduction in physical pain, a reduction in the severity of mental issues, a reduction in the amount of negative thoughts, a reduction in the amount of suicidal thoughts, a reduction in anxiety, a reduction in cancer cells, a reduction in infection, a reduction in frequency of psychotic episodes, a reduction in frequency of seizures, an improvement in the strength of muscles, an improvement in the strength of involuntary muscles, an improvement in the strength of the diaphragm, an improvement in the strength of the heart, an improvement in the strength of the digestive tract, or any combination thereof.

In some embodiments, safety may refer to the chances of an undesirable medical condition manifesting in a patient during or after a clinical intervention or a treatment. In some embodiments, safety may refer to the severity of an undesirable medical condition manifesting in a patient during or after a treatment. In some cases, an undesirable medical condition comprises a side effect, an adverse drug event, an undesirable surgical outcome, development of a secondary condition, loss of hair, loss of a limb, loss of tissue, loss of an organ, loss of blood, pain, death, changes in one or more biomarkers, or any combination thereof. In some embodiments, the treatment can cause or be associated with a new condition (e.g., a secondary condition due to the treatment) comprising a chronic condition, an infection, mental conditions, or an addiction.

In some embodiments, the treatment outcomes comprise an improvement of quality of life. In some embodiments, quality of life comprises the amount of physical activity by a subject, the amount of mental activity by a subject, self-reported levels of happiness from a subject, self-reported levels of well-being from a subject, amount of sleeping hours by a subject, amount of waking hours by a subject, amount of time a subject may spend with friends and family, amount of pain felt by a subject, or any combination thereof.

In some embodiments, the treatment outcomes are observed or self-reported by a subject. In some embodiments, the treatment outcomes are observed or reported by a caretaker or a health care provider.

In some embodiments, the treatment outcomes are obtained by performing a biomarker test on the treatment set of subjects and the reference set of subjects. In some embodiments, performing the biomarker test comprises obtaining biological samples from the treatment set of subjects and the reference set of subjects, and assaying the biological samples to determine the treatment outcomes. In some embodiments, the biomarker test comprises test or a measurement for a radiological test, a blood test, a urine test, a genetic test, an epigenomic test, gene expression test, protein expression test, or a metabolic biomarker test, a biopsy, a spinal tap, a tear test, a saliva test, or any combination thereof.

In some embodiments, a biomarker comprises a chemical biomarker, a genomic or epigenomic biomarker, a protein biomarker, a metabolic biomarker, or any combination thereof. In some embodiments, a biomarker comprises one or more biomolecules. The biomolecules can comprise an amino acid, a peptide, a protein, a nucleotide, a nucleic acid molecule, a simple sugar, a polysaccharide, or a lipid. In some embodiments, the nucleic acid comprises a single-stranded or a double-stranded nucleic acid. In some embodiments, the nucleic acid comprises DNA, DNA/RNA hybrid, or RNA, wherein the RNA comprises mRNA, tRNA, rRNA, cRNA, ncRNA, lncRNA, snoRNA, snRNA, piRNA, siRNA, or miRNA. In some embodiments, the biomarker provided herein comprises a complex of more than one types of biomolecules. For example, the biomarker can comprise a phosphorylated protein and nucleic acid complex or a methylated protein and nucleic acid complex. In some embodiments, the biomarker comprises a monosaccharide (e.g., glucose, fructose, galactose, etc.) a polysaccharide (e.g., glycogen), a lipid (e.g., a phospholipid, a fatty acid, a cholesterol, etc.), or a gas molecule (e.g., O₂, CO₂, NO, etc.). In some embodiments, the biomarker comprises a hormone, wherein the hormone comprises adrenaline, melatonin, noradrenaline, triiodothyronine, thyroxine, prostaglandin, leukotriene, prostacyclin, thromboxane, amylin, adrenocorticotropic hormone, angiotensinogen, vasopressin, atriopeptin, brain natriuretic, calcitonin, cholecystokinin, corticotropin-releasing hormone, cortistatin, enkephalin, endothelin, erythropoietin, galanin, gastrin, ghrelin, glucagon, gonadotropin-releasing hormone, growth hormone, hepcidin, gonadotropin, lactogen, inhibin, insulin, somatomedin, leptin, lipotropin, motilin, orexin, osteocalcin, parathyroid hormone, prolactin, relaxin, renin, secretinin, somatostatin, thrombopoietin, thyrotropin, guanylin, uroguanylin, serotonin, dopamine, oxytocin, endorphin, a steroid, testosterone, estrogen, dehydroepiandrosterone, androstenedione, dihydrotestosterone, aldosterone, estradiol, estrone, estriol, progesterone, calcitriol, or calcidiol. In some embodiments, the biomarker comprises sodium, potassium, magnesium, manganese, selenium, copper, chromium, fluoride, chloride, lithium, beryllium, calcium, bromide, iodide, acetone, methanol, ethanol, or any combination thereof.

In some embodiments, the treatment outcome provided herein comprises a clinical test result. In some embodiments, the clinical test result comprises amniocentesis, blood analysis, blood count, blood typing, bone marrow aspiration, cephalin-cholesterol flocculation, enzyme analysis, epinephrine tolerance test, glucose tolerance test, hematocrit, immunologic blood test, inulin clearance, serological test, thymol turbidity, gastric fluid analysis, kidney function test, liver function test, lumbar puncture, malabsorption test, Pap smear, phenolsulfonphthalein test, pregnancy test, prenatal testing, protein-bound iodine test, syphilis test, thoracentesis, thyroid function test, toxicology test, urinalysis/uroscopy, diagnostic imaging, angiocardiography, angiography, cerebral angiography, brain scanning, echoencephalography, magnetoencephalography, pneumoencephalography, cholecystography, echocardiography, endoscopic retrograde cholangiopancreatoscopy, lung ventilation/perfusion scan, magnetic resonance imaging, cardiac magnetic resonance imaging, functional magnetic resonance imaging, magnetic resonance spectroscopy, mammography, myelography, prenatal testing, tomography, computed tomography, positron emission tomography, single photon emission computed tomography, ultrasound, urography, genetic testing, complementation test, fluorescence in situ hybridization, preimplantation genetic diagnosis, measurement, ballistocardiography, electrocardiography, electroencephalography, electromyography, lumbar puncture, magnetic resonance spectroscopy, phonocardiography, pulmonary function test, semen analysis, physical and visual examination, auscultation, autopsy, biopsy, bronchoscopy, cardiac catheterization, colposcopy, Dick test, endoscopy, esophagogastroduodenoscopy, gynecological examination, laparoscopy, mediastinoscopy, nasopharyngolaryngoscopy, palpation, percussion, Rubin's test, semen analysis, skin test, patch test, Schick test, tuberculin test, toxicological examination, uroscopy, or any combination thereof.

### Generalized Pairwise Comparison (GPC) and Net Treatment Benefit (NTB)

In some embodiments, the method comprises comparing treatment outcomes between the first subject and the second subject for each of a plurality of clinical interventions and prioritizing or ranking the plurality of treatment outcomes for the subject. In some embodiments, the method comprises receiving user input of a prioritization function of the plurality of treatment outcomes provided herein. In some embodiments, the prioritization function is used to design a trial described herein.

In some embodiments, prioritizing or ranking comprises assigning a number to each clinical intervention or treatment outcome, wherein the number corresponds to a priority value or a rank value of each clinical intervention. In some embodiments, the number may be an integer. In some embodiments, no two clinical interventions are assigned the same number. In some embodiments, at least two clinical interventions may be assigned the same number. In some embodiments, prioritizing or ranking comprises assigning a plurality of numbers to each clinical intervention. In some embodiments, the prioritization function is selected by a subject. In some embodiments, the prioritization function is selected by a clinician. In some embodiments, the prioritization function is selected by a health care professional. In some embodiments, the prioritization function is selected by a patient. In some embodiments, the prioritization function is selected by a patient representative. In some embodiments, the prioritization function is selected by a member of the general public. In some embodiments, the prioritization function is selected based on at least one of efficacies, adverse effects, and/or thresholds of clinical relevance of individual treatment outcomes of the plurality of treatment outcomes. In some embodiments, the prioritization function comprises at least one of an ordering, a ranking, a set of weights, and a non-transitive ordering for individual treatment outcomes of the plurality of treatment outcomes.

In some embodiments, the set of weights comprises non-zero weights. In some embodiments, the set of weights comprises zero weights. In some embodiments, weights are expressed as a value between 0 and 1. In some embodiments, weights are expressed in percentages (e.g., with the sum of weights adding to 1).

In some embodiments, a non-transitive ordering may refer to an ordering with binary relations that are not transitive relations. In some embodiments, a non-transitive ordering may be synthesized from the plurality of treatment outcomes. In some embodiments, a non-transitive ordering may be synthesized from the plurality of treatment outcomes and a prioritization order or ranking of the plurality of treatment outcomes.

In some embodiments, the treatment outcomes comprise a plurality of endpoints. In some embodiments, the plurality of endpoints each comprise a measurement of a treatment progress or a treatment outcome. In some embodiments, the plurality of endpoints each comprise a projected treatment progress or a projected treatment outcome. In some embodiments, the plurality of endpoints each comprise one or more assays of a subject, a test of a subject, a medical examination of a subject, and any reports thereof. In some embodiments, each endpoint in the plurality of endpoints is created when a subject visits a hospital and receives a diagnostic, a consultation, a treatment, a therapy, or any combination thereof. In some embodiments, the plurality of endpoints is prioritized or ranked. In some embodiments, the plurality of endpoints is prioritized or ranked by a user.

In some embodiments, the method comprises generating a plurality of simulated trials, at least in part by: i) performing a set of pairwise comparisons between a first subject selected from the treatment set of subjects and a second subject selected from the reference set of subjects, based at least in part on the prioritization function, and ii) determining a net treatment benefit of the clinical intervention, based at least in part on the set of pairwise comparisons.

In some embodiments, the pairwise comparison comprises comparing the difference in the treatment outcomes between the first subject and the second subject. In some embodiments, the pairwise comparison comprises comparing the difference in the treatment outcomes between the first subject and the second subject to a clinical threshold. In some embodiments, the pairwise comparison comprises comparing each of a plurality of treatment outcomes between the first subject and the second subject. In some embodiments, the method comprises characterizing a pairwise comparison as a favorable, an unfavorable, or a tie comparison based at least in part on the difference in the treatment outcomes between the first subject and the second subject. In some embodiments, the pairwise comparison is characterized as a favorable, an unfavorable, or a tie based at least in part on the difference in the treatment outcomes being a positive difference greater than a threshold, a negative difference greater than a threshold, or a difference less than a threshold, respectively. In some embodiments, the method comprises determining a likelihood that the first subject has a better treatment outcome than the second subject, based at least in part on the set of pairwise comparisons. In some embodiments, the likelihood comprises a relative likelihood (e.g., a likelihood ratio or odds ratio, such as win ratio or success odds), an absolute likelihood, or a net likelihood (e.g., a difference between two likelihoods, such as net benefit). In some embodiments, the likelihood comprises a probability or a net probability (e.g., a difference between two probabilities).

In some embodiments, the clinical threshold comprises a minimal threshold for a positive outcome. In some embodiments, the clinical threshold comprises a minimal threshold for a clinically worthwhile effect. In some embodiments, the clinical threshold comprises an outcome of another treatment.

In some embodiments, the method further comprises selecting a clinical intervention from among a plurality of clinical interventions to be administered or provided to the subject, based at least in part on the net treatment benefit. In some embodiments, the method further comprises prescribing the clinical intervention to the subject based at least in part on the net treatment benefit. In some embodiments, the method further comprises administering or providing the clinical intervention to the subject based at least in part on the net treatment benefit.

In some embodiments, the set of pairwise comparisons comprises all possible pairwise combinations of a subject selected from the treatment set and a subject selected from the reference set. In some embodiments, the set of pairwise comparisons comprises all possible pairwise combinations of a subject selected from the treatment set and a subject selected from the treatment set. In some embodiments, the set of pairwise comparisons comprises all possible pairwise combinations of a subject selected from the treatment set plus the reference set and a subject selected from the treatment set plus the reference set.

In some embodiments, the net treatment benefit comprises a therapeutic benefit of the clinical intervention measured by a biomarker or observed by a person. In some embodiments, the net treatment benefit comprises self-reported satisfaction of a subject, reducing risk of a treatment, an increase in survivability, an increase in self-perceived well-being, an increase in well-being observed by a medical professional, an increase in self-perceived happiness, an increase in happiness observed by a medical professional, an increase in self-perceived mental health, an increase in mental health observed by a medical professional, an increase in physical activity, an increase in mental activity, an increase in sleep quality, an increase in metabolism, an increase in brain activity, an increase in hearing ability, an increase in sense of smell, an increase in visual acuity, an increase in the ability to taste, an increased cell regeneration, an increase in immune system strength, an increase in blood flow, an increased appetite, a reduction in pain, a reduction in physical pain, a reduction in the severity of mental issues, a reduction in the amount of negative thoughts, a reduction in the amount of suicidal thoughts, a reduction in anxiety, a reduction in cancer cells, a reduction in infection, a reduction in frequency of psychotic episodes, a reduction in frequency of seizures, an improvement in the strength of muscles, an improvement in the strength of involuntary muscles, an improvement in the strength of the diaphragm, an improvement in the strength of the heart, an improvement in the strength of the digestive tract, any other change in symptoms, clinical outcomes or biomarker values, or any combination thereof.

In some embodiments, the method comprises comparing the treatment outcomes between the first subject and the second subject at least in part by comparing a net benefit minus a net harm between the first subject and the second subject. In some embodiments, the net benefit comprises event-free survival time, progression-free survival time, overall survival time, efficacy, safety, quality of life, or any combination thereof.

In some embodiments, the net harm comprises an adverse event associated with the clinical intervention. The adverse event associated with the clinical intervention can comprise an adverse drug event, an allergy or reaction to the clinical intervention, a side effect of the clinical intervention, a toxicity of the clinical intervention, or any combination thereof. In some embodiments, a side effect comprises constipation, skin rash, dermatitis, diarrhea, dizziness, drowsiness, dry mouth, headache, insomnia, nausea, suicidal thoughts, abnormal heart rhythms, internal bleeding, fever, loss in appetite, increase in psychotic episodes, dissociative disorder, fatigue, swelling, allergic reactions, decrease in sex drive, infertility, menopause, alopecia, reduction in memory, reduction in attention span, hearing impairment, low blood platelet count, low red blood cell count, low white blood cell count, mucositis, moodiness, dry skin, erectile dysfunction, nerve damage, infection, or any combination thereof. In some cases, a toxicity comprises chemical toxicity, biological toxicity, physical toxicity, radiation toxicity, behavioral toxicity, or any combination thereof. In some embodiments, an adverse drug event comprises anaphylaxis, Stevens-Johnson Syndrome (SJS), toxic epidermal necrolysis (TEN), acute generalized exanthematous pustulosis (AGEP), and drug reaction with eosinophilia and systemic symptoms (DRESS).

### Subjects

In some embodiments, a subject comprises an individual being treated by a clinical intervention. In some embodiments, the subject comprises a patient with a disorder or disease or a healthy volunteer. In some embodiments, the patient comprises a past patient that was previously treated, a present patient that is being presently treated, a future patient that may receive treatment in the future, or combination thereof. In some cases, a subject may have one or more medical conditions. In some embodiments, the treatment set of subjects and/or the reference set of subjects comprise subjects having a disease or disorder. In some embodiments, the disease, the disorder, or the medical condition comprises a cancer, a leukemia, or a tumor. In some embodiments, the cancer, leukemia, or tumor is selected from the group consisting of acute lymphoblastic leukemia, acute myeloid leukemia, adrenocortical carcinoma, AIDS-related cancer, AIDS-related lymphoma, primary central nervous system lymphoma, anal cancer, appendix cancer, astrocytomas, rhabdoid tumor, basal cell carcinoma, bladder cancer, bone cancer, Ewing sarcoma, malignant fibrous histiocytoma, brain tumors, breast cancer, bronchial tumors, Burkitt lymphoma, carcinoid tumor, cardiac tumors, atypical teratoid tumor, central nervous system embryonal tumors, cervical cancer, cholangiocarcinoma, bile duct cancer, chordoma, chronic lymphocytic leukemia, chronic myelogenous leukemia, chronic myeloproliferative neoplasms, colorectal cancer, craniopharyngioma, cutaneous T-cell lymphoma, ductal carcinoma, medulloblastoma, endometrial cancer, uterine cancer, ependymoma, esophageal cancer, esthesioneuroblastoma, extracranial germ cell tumor, eye cancer, intraocular melanoma, retinoblastoma, fallopian tube cancer, gallbladder cancer, gastric cancer, gastrointestinal carcinoid tumor, gastrointestinal stromal tumors, germ cell tumors, childhood extracranial germ cell tumors, extragonadal germ cell tumors, ovarian germ cell tumors, testicular cancer, gestational trophoblastic disease, hairy cell leukemia, head and neck cancer, hepatocellular cancer, histiocytosis, Hodgkin lymphoma, pancreatic neuroendocrine tumors, Kaposi sarcoma, kidney cancer, Langerhans cell histiocytosis, laryngeal cancer, leukemia, lip and oral cavity cancer, lung cancer, non-small cell lung cancer, small cell lung cancer, tracheobronchial tumor, male breast cancer, melanoma, Merkel cell carcinoma, mesothelioma, metastatic cancer, metastatic squamous neck cancer, midline tract carcinoma, multiple endocrine neoplasia syndromes, multiple myeloma/plasma cell neoplasms, mycosis fungoides, myelodysplastic syndromes, myelodysplastic/myeloproliferative neoplasms, myelogenous leukemia, myeloid leukemia, myeloproliferative neoplasms, nasal cavity and paranasal sinus cancer, neuroblastoma, non-Hodgkin lymphoma, oral cancer, osteosarcoma, undifferentiated pleomorphic sarcoma, ovarian cancer, pancreatic cancer, islet cell tumors, papillomatosis, paraganglioma, paranasal sinus and nasal cavity cancer, parathyroid cancer, penile cancer, pharyngeal cancer, pheochromocytoma, pituitary tumor, plasma cell neoplasm/multiple myeloma, pleuropulmonary blastoma, pregnancy cancer, primary peritoneal cancer, prostate cancer, rectal cancer, recurrent cancer, retinoblastoma, rhabdomyosarcoma, salivary gland cancer, sarcoma, childhood rhabdomyosarcoma, childhood vascular tumors, Sezary syndrome, skin cancer, small intestine cancer, soft tissue sarcoma, squamous cell carcinoma of the skin, squamous neck cancer with occult primary, stomach cancer, t-cell lymphoma, testicular cancer, throat cancer, nasopharyngeal cancer, oropharyngeal cancer, hypopharyngeal cancer, thymoma and thymic carcinoma, thyroid cancer, tracheobronchial tumors, transitional cell cancer of the renal pelvis and ureter, urethral cancer, endometrial uterine cancer, uterine sarcoma, vaginal cancer, vascular tumors, vulvar cancer, and Wilms tumor.

In some embodiments, the disease, the disorder, or the medical condition comprises a genetic disorder selected from the group consisting of: 1p36 deletion syndrome, 1q21.1 deletion syndrome, 2q37 deletion syndrome, 5q deletion syndrome, 5,10-methenyltetrahydrofolate synthetase deficiency, 17q12 microdeletion syndrome, 17q12 microduplication syndrome, 18p deletion syndrome, 21-hydroxylase deficiency, Alpha 1-antitrypsin deficiency, AAA syndrome (achalasia-Addisonianism-alacrima syndrome), Aarskog-Scott syndrome, ABCD syndrome, Aceruloplasminemia, Acheiropodia, Achondrogenesis type II, achondroplasia, Acute intermittent porphyria, Adenylosuccinate lyase deficiency, Adrenoleukodystrophy, Alagille syndrome, ADULT syndrome, Aicardi-Goutières syndrome, Albinism, Alexander disease, Alfi's syndrome, alkaptonuria, Alport syndrome, Alternating hemiplegia of childhood, Amyotrophic lateral sclerosis—Frontotemporal dementia, Alström syndrome, Alzheimer's disease, Amelogenesis imperfecta, Aminolevulinic acid dehydratase deficiency porphyria, Androgen insensitivity syndrome, Angelman syndrome, Apert syndrome, Arthrogryposis-renal dysfunction-cholestasis syndrome, Ataxia telangiectasia, Axenfeld syndrome, Beare-Stevenson cutis gyrata syndrome, Beckwith-Wiedemann syndrome, Benjamin syndrome, biotinidase deficiency, Björnstad syndrome, Bloom syndrome, Birt-Hogg-Dubé syndrome, Brody myopathy, Brunner syndrome, CADASIL syndrome, Cat eye syndrome, CRASIL syndrome, Chronic granulomatous disorder, Campomelic dysplasia, Canavan disease, Carpenter Syndrome, CDKL5 deficiency disorder, Cerebral dysgenesis-neuropathy-ichthyosis-keratoderma syndrome (CEDNIK), Cystic fibrosis, Charcot-Marie-Tooth disease, CHARGE syndrome, Chediak-Higashi syndrome, Chondrodysplasia, Grebe type, Cleidocranial dysostosis, Cockayne syndrome, Coffin-Lowry syndrome, Cohen syndrome, collagenopathy, types II and XI, Congenital insensitivity to pain with anhidrosis (CIPA), Congenital Muscular Dystrophy, Comelia de Lange syndrome (CDLS), Cowden syndrome, CPO deficiency (coproporphyria), Cranio-lenticulo-sutural dysplasia, Cri du chat, Crohn's disease, Crouzon syndrome, Crouzonodermoskeletal syndrome (Crouzon syndrome with acanthosis nigricans), Currarino syndrome, Darier's disease, Dent's disease (Genetic hypercalciuria), Denys-Drash syndrome, De Grouchy syndrome, Down Syndrome, DiGeorge syndrome, Distal hereditary motor neuropathies, multiple types, Distal muscular dystrophy, Duchenne muscular dystrophy, Dravet syndrome, Edwards Syndrome, Ehlers-Danlos syndrome, Emanuel syndrome, Emery-Dreifuss syndrome, Epidermolysis bullosa, Erythropoietic protoporphyria, Fanconi anemia (FA), Fabry disease, Factor V Leiden thrombophilia, Fatal familial insomnia, Familial adenomatous polyposis, Familial dysautonomia, Familial Creutzfeld-Jakob Disease, Feingold syndrome, FG syndrome, Fragile X syndrome, Friedreich's ataxia, G6PD deficiency, Galactosemia, Gaucher disease, Gerstmann-Straussler-Scheinker syndrome, Gillespie syndrome, Glutaric aciduria, type I and type 2, GRACILE syndrome, Griscelli syndrome, Hailey-Hailey disease, Harlequin type ichthyosis, Hemochromatosis type 1, Hemochromatosis type 2A, Hemochromatosis type 2B, Haemochromatosis type 3, Hemochromatosis type 4, Hemochromatosis type 5, Hemophilia, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary spastic paraplegia (infantile-onset ascending hereditary spastic paralysis), Hermansky-Pudlak syndrome, Hereditary neuropathy with liability to pressure palsies (HNPP), Heterotaxy, Homocystinuria, Huntington's disease, Hunter syndrome, Hurler syndrome, Hutchinson-Gilford progeria syndrome, Hyperlysinemia, Hyperoxaluria, primary, Hyperphenylalaninemia, Hypoalphalipoproteinemia (Tangier disease), Hypochondrogenesis, Hypochondroplasia, Immunodeficiency-centromeric instability-facial anomalies syndrome (ICF syndrome), Incontinentia pigmenti, Ischiopatellar dysplasia, Isodicentric 15, Jackson-Weiss syndrome, Jacobsen syndrome, Joubert syndrome, Juvenile primary lateral sclerosis (JPLS), Keloid disorder, Kleefstra syndrome, Kniest dysplasia, Kosaki overgrowth syndrome, Krabbe disease, Kufor-Rakeb syndrome, LCAT deficiency, Lesch-Nyhan syndrome, Li-Fraumeni syndrome, Limb-Girdle Muscular Dystrophy, Lynch syndrome, lipoprotein lipase deficiency, Malignant hyperthermia, Maple syrup urine disease, Marfan syndrome, Maroteaux-Lamy syndrome, McCune-Albright syndrome, McLeod syndrome, MEDNIK syndrome, Mediterranean fever, familial, Menkes disease, Methemoglobinemia, Methylmalonic acidemia, Micro syndrome, Microcephaly, Miller-Dieker syndrome, Morquio syndrome, Mowat-Wilson syndrome, Muenke syndrome, Multiple endocrine neoplasia type 1 (Wermer's syndrome), Multiple endocrine neoplasia type 2, Muscular dystrophy, Muscular dystrophy, Duchenne and Becker type, Myostatin-related muscle hypertrophy, myotonic dystrophy, Natowicz syndrome, Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, Osteogenesis imperfecta, Pantothenate kinase-associated neurodegeneration, Patau syndrome (Trisomy 13), PCC deficiency (propionic acidemia), Porphyria cutanea tarda (PCT), Pendred syndrome, Peutz-Jeghers syndrome, Pfeiffer syndrome, Phelan-McDermid syndrome, Phenylketonuria, Pipecolic acidemia, Pitt-Hopkins syndrome, Polycystic kidney disease, Polycystic ovary syndrome (PCOS), Porphyria, Prader-Willi syndrome, Primary ciliary dyskinesia (PCD), Primary pulmonary hypertension, Protein C deficiency, Protein S deficiency, Proximal 18q deletion syndrome, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, Sjogren-Larsson syndrome, Spondyloepiphyseal dysplasia congenita (SED), Shprintzen-Goldberg syndrome, Sickle cell anemia, Siderius X-linked mental retardation syndrome, Sideroblastic anemia, Sly syndrome, Smith-Lemli-Opitz syndrome, Smith-Magenis syndrome, Snyder-Robinson syndrome, Spinal muscular atrophy, Spinocerebellar ataxia (types 1-29), SSB syndrome (SADDAN), Stargardt disease (macular degeneration), Stickler syndrome (multiple forms), Strudwick syndrome (spondyloepimetaphyseal dysplasia, Strudwick type), Tay-Sachs disease, Tetrahydrobiopterin deficiency, Thanatophoric dysplasia, Treacher Collins syndrome, Tuberous sclerosis complex (TSC), Turner syndrome, Usher syndrome, Variegate porphyria, von Hippel-Lindau disease, von Willebrand disease, Waardenburg syndrome, Warkany syndrome 2, Weissenbacher-Zweymuller syndrome, Williams syndrome, Wilson disease, Woodhouse-Sakati syndrome, Wolf-Hirschhorn syndrome, Xeroderma pigmentosum, X-linked intellectual disability and macroorchidism (fragile X syndrome), X-linked spinal-bulbar muscle atrophy (spinal and bulbar muscular atrophy), Xp11.2 duplication syndrome, X-linked severe combined immunodeficiency (X-SCID), X-linked sideroblastic anemia (XLSA), 47,XXX (triple X syndrome), XXXX syndrome (48, XXXX), XXXXX syndrome (49,XXXXX), XXXXY syndrome (49,XXXXY), XYY syndrome (47,XYY), XXYY syndrome (48,XXYY), XYYY syndrome (48,XYYY), XXXY syndrome (48,XXXY), XYYYY syndrome (49,XYYYY), and Zellweger syndrome.

In some embodiments, the disease, the disorder, or the medical condition comprises septicemia, heart failure, osteoarthritis, pneumonia, diabetes, acute myocardial infraction, cardiac dysrhythmia, chronic obstructive pulmonary disease, bronchiectasis, mood disorder, implant complications, graft complications, coronary atherosclerosis, cardiovascular disease, or any combination thereof. In some embodiments, the disease, the disorder, or the medical condition is an adult disease, disorder, or medical condition. In some embodiments, the disease, the disorder, or the medical condition is a child disease, disorder, or medical condition.

In some embodiments, subjects of a clinical trial comprise a treatment set of subjects and a reference set of subjects. In some embodiments, the treatment set of subjects receives the clinical intervention and the reference set of subjects does not receive the clinical intervention. In some embodiments, the treatment set of subjects and the reference set of subjects both receive the clinical intervention. In some embodiments, the reference set of subjects receives a standard of care therapy.

In some embodiments, the first subject is randomly selected from the treatment set of subjects, and wherein the second subject is randomly selected from the reference set of subjects.

In some embodiments, the treatment set of subjects comprise at least 1 subject, at least 2 subjects, at least 3 subjects, at least 4 subjects, at least 5 subjects, at least 6 subjects, at least 7 subjects, at least 8 subjects, at least 9 subjects, at least 10 subjects, at least 50 subjects, at least 100 subjects, at least 150 subjects, at least 200 subjects, at least 250 subjects, at least 300 subjects, at least 350 subjects, at least 400 subjects, at least 450 subjects, at least 500 subjects, at least 550 subjects, at least 600 subjects, at least 650 subjects, at least 700 subjects, at least 750 subjects, at least 800 subjects, at least 850 subjects, at least 900 subjects, at least 950 subjects, at least 1000 subjects, at least 2000 subjects, at least 3000 subjects, at least 4000 subjects, at least 5000 subjects, at least 6000 subjects, at least 7000 subjects, at least 8000 subjects, at least 9000 subjects, at least 10000 subjects, at least 20000 subjects, at least 30000 subjects, at least 40000 subjects, at least 50000 subjects, at least 60000 subjects, at least 70000 subjects, at least 80000 subjects, at least 90000 subjects, at least 100000 subjects, at least 200000 subjects, at least 300000 subjects, at least 400000 subjects, at least 500000 subjects, at least 600000 subjects, at least 700000 subjects, at least 800000 subjects, at least 900000 subjects, at least 1000000 subjects, at least 2000000 subjects, at least 3000000 subjects, at least 4000000 subjects, at least 5000000 subjects, at least 6000000 subjects, at least 7000000 subjects, at least 8000000 subjects, at least 9000000 subjects, or at least 10000000 subjects. In some embodiments, the reference set of subjects each comprise at least 1 subject, at least 2 subjects, at least 3 subjects, at least 4 subjects, at least 5 subjects, at least 6 subjects, at least 7 subjects, at least 8 subjects, at least 9 subjects, at least 10 subjects, at least 50 subjects, at least 100 subjects, at least 150 subjects, at least 200 subjects, at least 250 subjects, at least 300 subjects, at least 350 subjects, at least 400 subjects, at least 450 subjects, at least 500 subjects, at least 550 subjects, at least 600 subjects, at least 650 subjects, at least 700 subjects, at least 750 subjects, at least 800 subjects, at least 850 subjects, at least 900 subjects, at least 950 subjects, at least 1000 subjects, at least 2000 subjects, at least 3000 subjects, at least 4000 subjects, at least 5000 subjects, at least 6000 subjects, at least 7000 subjects, at least 8000 subjects, at least 9000 subjects, at least 10000 subjects, at least 20000 subjects, at least 30000 subjects, at least 40000 subjects, at least 50000 subjects, at least 60000 subjects, at least 70000 subjects, at least 80000 subjects, at least 90000 subjects, at least 100000 subjects, at least 200000 subjects, at least 300000 subjects, at least 400000 subjects, at least 500000 subjects, at least 600000 subjects, at least 700000 subjects, at least 800000 subjects, at least 900000 subjects, at least 1000000 subjects, at least 2000000 subjects, at least 3000000 subjects, at least 4000000 subjects, at least 5000000 subjects, at least 6000000 subjects, at least 7000000 subjects, at least 8000000 subjects, at least 9000000 subjects, or at least 10000000 subjects.

In some embodiments, the treatment set of subjects and the reference set of subjects each comprise at least 1 subject, at least 2 subjects, at least 3 subjects, at least 4 subjects, at least 5 subjects, at least 6 subjects, at least 7 subjects, at least 8 subjects, at least 9 subjects, at least 10 subjects, at least 50 subjects, at least 100 subjects, at least 150 subjects, at least 200 subjects, at least 250 subjects, at least 300 subjects, at least 350 subjects, at least 400 subjects, at least 450 subjects, at least 500 subjects, at least 550 subjects, at least 600 subjects, at least 650 subjects, at least 700 subjects, at least 750 subjects, at least 800 subjects, at least 850 subjects, at least 900 subjects, at least 950 subjects, at least 1000 subjects, at least 2000 subjects, at least 3000 subjects, at least 4000 subjects, at least 5000 subjects, at least 6000 subjects, at least 7000 subjects, at least 8000 subjects, at least 9000 subjects, at least 10000 subjects, at least 20000 subjects, at least 30000 subjects, at least 40000 subjects, at least 50000 subjects, at least 60000 subjects, at least 70000 subjects, at least 80000 subjects, at least 90000 subjects, at least 100000 subjects, at least 200000 subjects, at least 300000 subjects, at least 400000 subjects, at least 500000 subjects, at least 600000 subjects, at least 700000 subjects, at least 800000 subjects, at least 900000 subjects, at least 1000000 subjects, at least 2000000 subjects, at least 3000000 subjects, at least 4000000 subjects, at least 5000000 subjects, at least 6000000 subjects, at least 7000000 subjects, at least 8000000 subjects, at least 9000000 subjects, or at least 10000000 subjects.

### Computer Systems and Computer-Readable Media

In some aspects, the present disclosure describes a computer-implemented system for designing a trial evaluating a clinical intervention in a subject, comprising: a database configured to store a plurality of treatment outcomes for a treatment set of subjects and a reference set of subjects, wherein the treatment set of subjects receives the clinical intervention, and wherein the reference set of subjects does not receive the clinical intervention; and one or more computer processors operatively coupled to the database, wherein the one or more computer processors are individually or collectively programmed to: receive user input of a prioritization function of the plurality of treatment outcomes; generate a plurality of simulated trials, at least in part by: i) performing a set of pairwise comparisons between a first subject selected from the treatment set of subjects and a second subject selected from the reference set of subjects, based at least in part on the prioritization function, and ii) determining a net treatment benefit of the clinical intervention, based at least in part on the set of pairwise comparisons; and determine a parameter of the trial based at least in part on the plurality of simulated trials.

In another aspect, the present disclosure provides a computer-implemented system for evaluating treatment outcomes of a clinical intervention in a subject, comprising: a) a database configured to store a plurality of treatment outcomes for a treatment set of subjects and a reference set of subjects, wherein the treatment set of subjects receives the clinical intervention, and wherein the reference set of subjects does not receive the clinical intervention; and b) one or more computer processors operatively coupled to the database, wherein the one or more computer processors are individually or collectively programmed to: i) receive user input on a set of pairwise clinical scenarios, and ii) generate a prioritization function of the plurality of treatment outcomes, based at least in part on the user input.

In some embodiments, a computing system comprises a computer device, including but not limited to a mobile computer, smartphone, smartwatch, tablet, electronic notebook, laptop, or any combination thereof.

In some embodiments, the one or more computer processors are individually or collectively programmed to further characterize a pairwise comparison as a favorable, an unfavorable, or a tie comparison based at least in part on the difference in the treatment outcomes between the first subject and the second subject. In some embodiments, a pairwise comparison is characterized as a favorable, an unfavorable, or a tie comparison based at least in part on the difference in the treatment outcomes being a positive difference greater than a threshold, a negative difference greater than a threshold, or a difference less than a threshold, respectively.

In some embodiments, the one or more computer processors are individually or collectively programmed to further determine a likelihood that the first subject has a better treatment outcome than the second subject, based at least in part on the set of pairwise comparisons. In some embodiments, the computer system comprises comparing the treatment outcomes between the first subject and the second subject at least in part by comparing a net benefit minus a net harm between the first subject and the second subject.

In some embodiments, the one or more computer processors are individually or collectively programmed to further compare treatment outcomes between the first subject and the second subject for each of a plurality of clinical interventions, and prioritizing or ranking the plurality of clinical interventions for the subject.

In some embodiments, the one or more computer processors are individually or collectively programmed to model a set of variables of the plurality of simulated trials. In some embodiments, the variables of the plurality of simulated trials comprise a sample size, endpoint, randomization, power, dropout rate, dependency of treatment outcomes, variance of treatment outcomes, or minimal detectable treatment outcome. In some embodiments, the variables of the plurality of simulated trials comprise a dropout rate of the plurality of simulated trials, a dependency of treatment outcomes, a variance of treatment outcomes, or a minimal detectable treatment outcome. In some embodiments, the one or more computer processors are individually or collectively programmed to model the dependency of treatment outcomes of the plurality of simulated trials. In some embodiments, modeling the dependency of the treatment outcomes of simulated trials comprises modeling based on user input on the dependency of treatment outcomes or modeling based on historical data.

In some embodiments, the database of the computer-implemented system is configured to store user input or historical data on the dependency of treatment outcomes.

In some embodiments, the one or more computer processors are individually or collectively programmed to determine a parameter of the trial based at least in part on the plurality of simulated trials. In some embodiments, the parameters of the trial comprise an objective, type of study design, study group, study population, sample size, intervention, endpoint, outcome, randomization, blinding, data collection, data cleaning, data storage, statistical test, power, significance level, missing data, dropout rate, or any combination thereof. In some embodiments, the method provided herein comprises determining an endpoint, sample size, or power.

In some aspects, the present disclosure describes a non-transitory computer-readable medium comprising machine-executable code that, upon execution by one or more computer processors, implements a method for designing a trial evaluating a clinical intervention in a subject, the method comprising: obtaining a dataset for a treatment set of subjects and a reference set of subjects, wherein the treatment set of subjects receives the clinical intervention and the reference set of subjects does not receive the clinical intervention, and wherein the dataset comprises a plurality of treatment outcomes for the treatment set of subjects and the reference set of subjects, receiving user input of a prioritization function of the plurality of treatment out-comes; generating a plurality of simulated trials, at least in part by: i) performing a set of pairwise comparisons between a first subject selected from the treatment set of subjects and a second subject selected from the reference set of subjects, based at least in part on the prioritization function, and ii) determining a net treatment benefit of the clinical intervention, based at least in part on the set of pairwise comparisons; and d) determining a parameter of the trial based at least in part on the plurality of simulated trials.

Another aspect of the present disclosure provides a non-transitory computer-readable medium comprising machine-executable code that, upon execution by one or more computer processors, implements a method for evaluating treatment outcomes of a clinical intervention in a subject, the method comprising: a) obtaining a dataset for a treatment set of subjects and a reference set of subjects, wherein the treatment set of subjects receives the clinical intervention and the reference set of subjects does not receive the clinical intervention, and wherein the dataset comprises a plurality of treatment outcomes for the treatment set of subjects and the reference set of subjects; b) receiving user input on a set of pairwise clinical scenarios; and c) generating a prioritization function of the plurality of treatment outcomes, based at least in part on the user input.

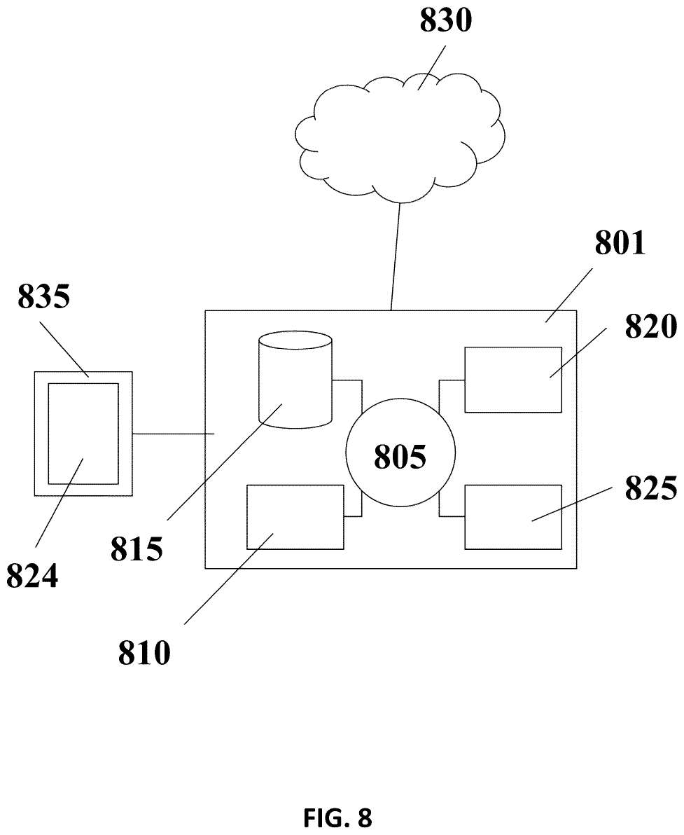

The present disclosure provides computer systems that are programmed to implement methods of the disclosure. FIG. 8 shows a computer system 801 that is programmed or otherwise configured to, for example, obtain datasets and treatment outcomes, receive user input of a prioritization function, generate a plurality of simulated trials, perform pairwise comparisons of treatment outcomes between subjects, identify a net treatment benefit of a clinical intervention for a subject, and determine a parameter of the trial based at least in part on the plurality of simulated trials.

The computer system 801 may regulate various aspects of analysis, calculation, and generation of the present disclosure, such as, for example, obtaining datasets and treatment outcomes, receiving user input of a prioritization function, generating a plurality of simulated trials, performing pairwise comparisons of treatment outcomes between subjects, identifying a net treatment benefit of a clinical intervention for a subject, and determining a parameter of the trial based at least in part on the plurality of simulated trials. The computer system 801 may be an electronic device of a user or a computer system that is remotely located with respect to the electronic device. The electronic device may be a mobile electronic device.

The computer system 801 includes a central processing unit (CPU, also “processor” and “computer processor” herein) 805, which may be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 may be a data storage unit (or data repository) for storing data. The computer system 801 may be operatively coupled to a computer network (“network”) 830 with the aid of the communication interface 820. The network 830 may be the Internet, an internet and/or extranet, or an intranet and/or extranet that is in communication with the Internet.

The network 830 in some cases is a telecommunication and/or data network. The network 830 may include one or more computer servers, which may enable distributed computing, such as cloud computing. For example, one or more computer servers may enable cloud computing over the network 830 (“the cloud”) to perform various aspects of analysis, calculation, and generation of the present disclosure, such as, for example, identifying a net treatment benefit of a clinical intervention for a subject, obtaining datasets and treatment outcomes, receiving user input of a prioritization function, and performing pairwise comparisons of treatment outcomes between subjects. Such cloud computing may be provided by cloud computing platforms such as, for example, Amazon Web Services (AWS), Microsoft Azure, Google Cloud Platform, and IBM cloud. The network 830, in some cases with the aid of the computer system 801, may implement a peer-to-peer network, which may enable devices coupled to the computer system 801 to behave as a client or a server.

The CPU 805 may comprise one or more computer processors and/or one or more graphics processing units (GPUs). The CPU 805 may execute a sequence of machine-readable instructions, which may be embodied in a program or software. The instructions may be stored in a memory location, such as the memory 810. The instructions may be directed to the CPU 805, which may subsequently program or otherwise configure the CPU 805 to implement methods of the present disclosure. Examples of operations performed by the CPU 805 may include fetch, decode, execute, and writeback.

The CPU 805 may be part of a circuit, such as an integrated circuit. One or more other components of the system 801 may be included in the circuit. In some cases, the circuit is an application specific integrated circuit (ASIC).

The storage unit 815 may store files, such as drivers, libraries and saved programs. The storage unit 815 may store user data, e.g., user preferences and user programs. The computer system 801 in some cases may include one or more additional data storage units that are external to the computer system 801, such as located on a remote server that is in communication with the computer system 801 through an intranet or the Internet.

The computer system 801 may communicate with one or more remote computer systems through the network 830. For instance, the computer system 801 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 801 via the network 830.

Methods as described herein may be implemented by way of machine (e.g., computer processor) executable code stored on an electronic storage location of the computer system 801, such as, for example, on the memory 810 or electronic storage unit 815. The machine executable or machine-readable code may be provided in the form of software. During use, the code may be executed by the processor 805. In some cases, the code may be retrieved from the storage unit 815 and stored on the memory 810 for ready access by the processor 805. In some situations, the electronic storage unit 815 may be precluded, and machine-executable instructions are stored on memory 810.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 801, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 may include or be in communication with an electronic display 835 that comprises a user interface (UI) 840 for providing, for example, a clinical intervention for a subject. Examples of Us include, without limitation, a graphical user interface (GUI) and web-based user interface.

Methods and systems of the present disclosure may be implemented by way of one or more algorithms. An algorithm may be implemented by way of software upon execution by the central processing unit 805. The algorithm can, for example, identify a net treatment benefit of a clinical intervention for a subject, obtain datasets and treatment outcomes, receive user input of a prioritization function, and perform pairwise comparisons of treatment outcomes between subjects.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### EXAMPLES

The following examples are provided to further illustrate some embodiments of the present disclosure, but are not intended to limit the scope of the disclosure; it will be understood by their exemplary nature that other procedures, methodologies, or techniques may alternatively be used.

### Example 1

This example demonstrates a method for designing a clinical trial evaluating a clinical intervention in trial subjects. The example focuses on the design of a clinical trial using generalized pairwise comparisons to test a less intensive treatment regimen for acute promyelocytic leukemia (APL).

Showing “similar efficacy” of a less intensive treatment may require a non-inferiority trial. Yet such clinical trials may be challenging to design and conduct. In APL, great progress has been achieved with the introduction of targeted therapies, but toxicity remains a major clinical issue. There is a pressing need to show the favorable benefit/risk of less intensive treatment regimens. Provided herein is a design of a clinical trial that uses generalized pairwise comparisons of five prioritized outcomes (alive and event-free at 2 years, grade 3/4 documented infections, differentiation syndrome, hepatotoxicity, and neuropathy) to confirm a favorable benefit/risk of a less intensive treatment regimen. Simulations were conducted based on historical data and assumptions about the differences expected between the standard of care and the less intensive treatment regimen to calculate the sample size required to have high power to show a positive Net Treatment Benefit in favor of the less intensive treatment regimen. Across 10,000 simulations, average sample sizes of 260 to 300 patients are required for a trial using generalized pairwise comparisons to detect typical Net Treatment Benefits of 0.19 (interquartile range 0.14-0.23 for a sample size of 280). The Net Treatment Benefit is interpreted as a difference between the probability of doing better on the less intensive treatment regimen than on the standard of care, minus the probability of the opposite situation. A Net Treatment Benefit of 0.19 translates to a number needed to treat of about 5.3 patients (1/0.19˜5.3). Generalized pairwise comparisons allow for simultaneous assessment of efficacy and safety, with priority given to the former. The sample size required may be of the order of 300 patients, as compared with more than 700 patients for a non-inferiority trial using a margin of 4% against the less intensive treatment regimen for the absolute difference in event-free survival at 2 years, as considered here.

### Introduction

In many clinical situations, especially in oncology, the standard of care has known efficacy but also untoward effects that make the treatment difficult to tolerate at full dose and schedule. A commonly used strategy is to start therapy at the recommended dose and schedule, and to reduce the dose or extend the schedule when adverse events occur. However, it may be desirable to use a less intensive treatment strategy upfront in frail populations, such as elderly patients, heavily pretreated patients, or patients with comorbidities. In addition, conventional dose-finding trial designs in oncology are based on the concept of maximum tolerated dose, which may identify a dose higher than the most effective dose, especially for targeted anticancer drugs and biologicals. In such cases, the lower dose may be as effective and have less toxicity. For these reasons, it is often necessary to compare a less intensive therapy with the standard therapy, with the hope of keeping similar efficacy while improving the treatment tolerability. Improvements in treatment tolerability can be shown in superiority trial designs, while showing “similar efficacy” of a less intensive treatment requires non-inferiority (NI) trial designs.

A large amount of literature has been devoted to NI trial designs and their inherent difficulties. In most cases, NI trials have no internal check of “assay sensitivity,” the capacity of the trial to distinguish between an effective and an ineffective therapy. An NI trial without assay sensitivity may in fact declare NI if both treatments are equally ineffective. NI trials are biased toward NI if they are poorly designed and conducted (e.g., due to lack of blinding). All aspects of trial conduct that favor equality between the two randomized treatment groups, for example, a large amount of treatment discontinuations or of patients lost to follow-up, bias the results of an NI trial toward a favor-able conclusion of NI. Yet, a review by Wangge et al. (Room for improvement in conducting and reporting non-inferiority randomized controlled trials on drugs: a systematic review, which publication is hereby incorporated by reference in its entirety for all purposes.) showed that elementary precautions against bias are not always implemented in practice; for instance, more than a third of the 232 considered NI trials registered in PubMed did not use blinding. NI trials also rely on an assumption of constancy: the conditions under which the control arm (here, the more intensive treatment) was shown to have efficacy are supposed to still hold. The study of Wangge et al. reported that less than 5% of the NI trials explicitly reported the similarity between the trial and the previous comparator's trials. Another major difficulty for NI trials resides in the pre-specification of an NI margin: no consensus exists on the choice of the NI margin, and recommendations differ between agencies, for instance between the US Food and Drug Administration and the European Medicines Agency. Undue emphasis on a statistical calculation of the NI margin leads to complicated methods that depend crucially on the availability of historical data, while an undue emphasis on clinical relevance may lead to “cherry-picking” that leads to attainable sample sizes. In fact, many NI trials do not provide any details on the NI margin. Studies in different settings have shown that only between 20% and 46% of NI trials report the justification for the choice of the NI margin. In addition, most trials do not report the method of determination of the NI margin in a way that allows replicability. An additional difficulty with NI trials is that they impact on evolving standards of care, thereby opening the gate to so-called “biocreep”, which refers to the cyclical phenomenon where a slightly inferior treatment becomes the active control for the next generation of NI trials, which over time may lead to degradation of the efficacy of the standards of care. Moreover, the sample sizes required for NI trials may be larger than those required to show superiority because the NI margin may be smaller than the effect one may wish to detect in a superiority trial, and the experimental treatment may be expected to be slightly inferior to the current standard of care-especially if the experimental treatment is a lower dose or a less intensive schedule of the standard of care, as is our focus here. A review of NI trials of reduced intensity therapies indeed suggested that fewer of these trials demonstrated NI or superiority, as compared with other NI trials. In fact, the small deficits expected of reduced intensity therapies may not justify the very large sample sizes that may be required for formal statistical tests of NI, especially in oncology, where the gains in toxicity may have a substantial impact on the patients' quality of life.

For all these reasons, NI trials may in some situations be forgone even when there is a pressing need for less intensive treatment schedules. Such is the case in acute promyelocytic leukemia (APL), a rare form of acute myeloid leukemia in which great progress has been achieved since the introduction of targeted therapies, such as all-trans retinoic acid (ATRA) and arsenic trioxide. This progress allowed for decreased reliance on the previous standard of intense chemotherapy as frontline treatment for APL, with attempts to reduce treatment intensity and enhance tolerability. Of note, and despite a generally improved tolerability from targeted therapy, toxicity remains a problem, particularly the “differentiation syndrome” associated with the use of ATRA. In APL, several phase 3 trials have used an NI design. Nevertheless, when NI trials are used, “similar efficacy” is usually achieved at the expense of not formally testing the superiority of the tolerability outcomes which often motivate the trial. An alternative approach may be to conduct a superiority trial for tolerability endpoints, with efficacy outcomes presented in secondary analyses. This approach is seldom used in oncology, due to concerns with loss of efficacy, but in one case, it has been used in APL with quality of life as the primary endpoint. Presented herein is an alternative to the problem of showing similar efficacy and better tolerability. The methods described herein uses generalized pairwise comparisons of prioritized outcomes to formally analyze efficacy and safety outcomes using a single test statistic. Specifically, the clinical situation addressed here concerns the design of a randomized trial for patients with APL, with the explicit aim of ensuring sufficient power and a single hypothesis testing framework for efficacy and safety outcomes of interest, given the interest of comparing a reduced dose of ATRA with the standard dose.

### Methods

### 1. Generalized Pairwise Comparisons and Net Treatment Benefit.

The method of generalized pairwise comparisons of prioritized outcomes was developed to analyze simultaneously as many outcomes of any type as desired, as long as it is possible to ascertain, given a pair of patients taken at random from each group, which of these two patients has a more desirable outcome. For a single outcome, a pair is considered “favorable” if the patient treated with the experimental agent has a better outcome than the control patient, “unfavorable” if the control patient has a better outcome, and a “tie” if the outcomes are clinically similar. For multiple outcomes, the idea involves prioritizing the outcomes from the most important to the least important and to perform pairwise comparisons on outcomes of successively lower priority until the pair can be classified as favorable or unfavorable. The order of priority may be arbitrary, but may be based on clinical judgment, regulatory precedent, and additional scientific considerations. One measure of treatment effect when using generalized pairwise comparisons is called the Net Treatment Benefit (NTB), defined as the population equivalent of the difference between the proportion of favorable pairs and the proportion of unfavorable pairs. When the only outcome of interest is a time to event, for example, event-free survival (EFS), the NTB is the net chance of a better EFS. For multiple outcomes, for example, EFS and Grade 3 or 4 toxicities, the NTB captures the net chance of a better EFS or less toxicity.

Assume one wishes to compare a group of patients receiving some intervention (called “treatment”) with a group of patients receiving standard of care (called “control”). Denoted the outcome of interest X and Y in the treatment group and the control group, respectively. Pairwise comparisons are carried out by forming all possible pairs of patients, taking one patient from each group. (Buyse M. Generalized pairwise comparisons of prioritized outcomes in the two-sample problem. Stat Med 2010; 29: 3245-3257, which publication is hereby incorporated by reference in its entirety for all purposes). A pairwise score is defined as follows for the i^{th} patient in the treatment group and the j^{th} patient in the control group:

$${u}_{i j} = \{ \begin{matrix}+ 1 if {X}_{i} ≻ {Y}_{j} \\ - 1 if {X}_{i} ≺ {Y}_{j} \\ 0 otherwise\end{matrix}$$

where the symbols “>” and “<” denote superiority and inferiority, respectively. The concepts of superiority and inferiority depend on the type of variable considered. They can easily be defined for binary variables (for instance, treatment success/failure), ordered categorical variables (for instance, none, mild, moderate, or severe toxicity), continuous variables (for instance, left ventricular ejection fraction, with a larger value being preferable), or time to event variables (for instance, overall survival, with larger values being preferable). The pairwise score uᵢⱼ is equal to 1 if the pair favors treatment (“favorable” or “win”), to −1 if the pair favors control (“unfavorable” or “loss”), and to 0 if the pair favors neither treatment nor control (“tie”) (Pocock S J, Ariti C A, Collier T J, Wang D. The win ratio: a new approach to the analysis of composite endpoints in clinical trials based on clinical priorities. European Heart Journal 2012; 33: 176-82, which publication is hereby incorporated by reference in its entirety for all purposes).
2. Prioritized outcomes. The outcomes of interest in the present situation were, in order of decreasing priority, and with the favorable outcome shown first within parenthesis:
1) Event free survival (EFS), considered a binary outcome at 2 years of follow-up (alive and event-free at 2 years versus in relapse or dead by 2 years)
2) Grade 3/4 documented infections (no versus yes)
3) Grade 3/4 differentiation syndrome (no versus yes)
4) Grade 3/4 hepatotoxicity (no versus yes)
5) Grade 3/4 neuropathy (no versus yes)

Grade 3 and Grade 4 toxicity denote severe and life-threatening events, respectively. By selecting EFS as the first priority, pairs of patients are compared on toxicity outcomes only if the comparison for EFS results in a tie (when both patients of the pair are either alive and event-free at 2 years or in relapse or dead by 2 years). If the EFS values differ for a pair of patients, toxicity comparisons are not performed. This prioritized outcome approach explicitly accounts for any potential degradation of the EFS outcome in the experimental arm. The order of priority chosen for the toxicity outcomes reflects the clinical importance of these outcomes in terms of morbidity, duration of hospitalization, risk of complications, such as infections, inconvenience to the patient, and cost.

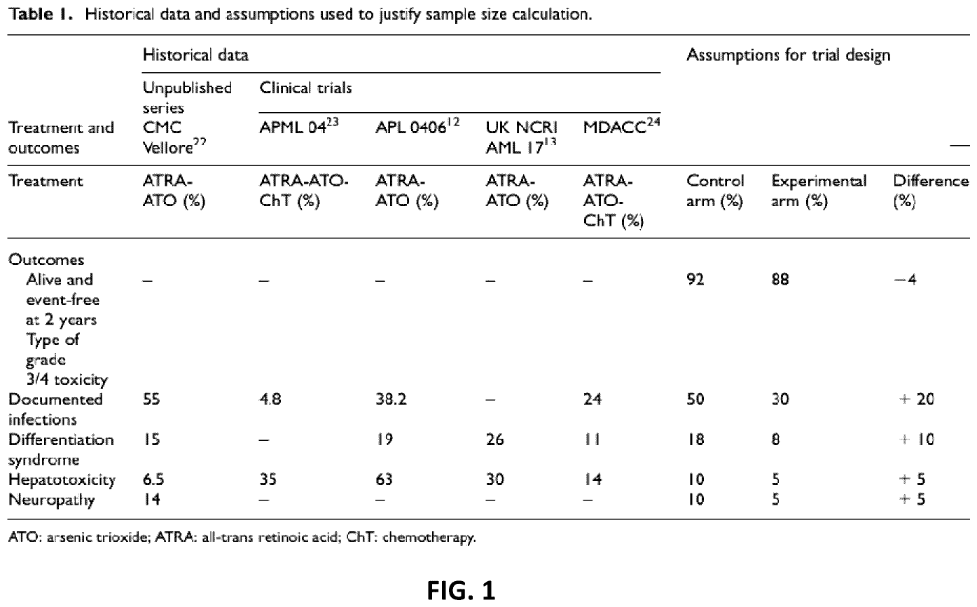

Assume that there are two outcomes of interest, which can be prioritized because an improvement in one of these outcomes is preferred to an improvement in the other outcome. The outcomes are again denoted X and Y in the treatment and the control groups, respectively, with priorities denoted by subscripts 1 or 2 (1 being higher priority than 2). The pairwise score is defined as follows for the i^{th} patient in the experimental group and the j^{th} patient in the control group:

$${u}_{i j} = \{ \begin{matrix}+ 1 if {X}_{1 , i} ≻ {Y}_{1 , j} or ( {X}_{1 , i} ≍ {Y}_{1 , j} and {X}_{2 , i} ≻ {Y}_{2 , j} ) \\ - 1 if {X}_{1 , i} ≺ {Y}_{1 , j} or ( {X}_{1 , i} ≍ {Y}_{1 , j} and {X}_{2 , i} ≺ {Y}_{2 , j} ) \\ 0 otherwise\end{matrix}$$

where the symbols “>” and “<” denote superiority and inferiority, respectively, and =denotes neutrality on the scale of the variables. The pairwise score generalizes easily to any number of prioritized outcomes of any type. The advantage of using prioritized outcomes, besides intuitive appeal, is that the correlation between the outcomes is taken into account (Buyse et al., 2021. The net benefit of a treatment should take the correlation between benefits and harms into account. J Clin Epidemiol 2021; 137: 148-158, which publication is hereby incorporated by reference in its entirety for all purposes).
3. Historical data and assumptions for trial design. The trial may compare an experimental treatment (reduced dose of ATRA) with control (standard dose). Parameters used for sample size calculation using generalized pairwise comparisons required assumptions obtained from historical sources. Table 1, as shown in FIG. 1, shows data on the grade 3/4 types of toxicity from a published cohort of patients treated with ATRA at the Christian Medical Center of Vellore (India) between January 2015 and May 2020, 22 and from four published randomized clinical trials.

Based on historical data shown in Table 1 (FIG. 1), assumptions for the experimental and control arms were elicited from clinicians with expertise in APL (M.S. and V.M.). The right-hand side of Table 1 (FIG. 1) summarizes these assumptions.

### 4. Testing Procedure for NTB.

The hypothesis of interest in this trial is:
H0: NTB_{Red.}=0 vs HA: NTB_{Red.}≠0
where NTB_{Red.} is the NTB of the reduced ATRA dose as compared with the standard dose. The variance of the test statistic required for the test was computed via the asymptotic distribution of the NTB estimator, using U-statistics theory. The procedure is implemented in the R package “BuyseTest,” freely available on GitHub and CRAN.26

### 5. Simulations Using Multivariate Distributions

Sample size calculations were performed via simulations. These consisted of generating a large number of trials (10,000) with increasing sample sizes, equally divided between the two treatment groups. For each simulated trial, a p-value was computed based on the asymptotic distribution of the test statistic. The power of the test was calculated as the empirical rejection rate among the simulated trials, with a two-sided α-level of 5%. The sample size for the trial was chosen to provide a power of at least 80%.

As the procedure accounted for multiple outcomes simultaneously, it was important to account for the dependencies between these outcomes. Patient-level data of the CMC Vellore trial was used, to estimate a 5×5 odds ratio matrix, where each entry (k, l) corresponded to the estimated odds ratio between outcomes “k” and “l”. This measure of association between all pairs of outcomes was used to simulate data from a multivariate Bernoulli distribution, with marginal probabilities corresponding to the desired target for each treatment arm (see Table 1) and dependencies corresponding as close as possible to the odds ratio matrix. The R package “mipfp” was utilized, which is freely available on CRAN, to implement a so-called iterative proportional fitting procedure for this task.
6. Dropouts. The trial design assumed a 10% dropout rate within 2 years. Dropouts only affected EFS because the toxicity outcomes may be observed very early in the trial, and it was therefore assumed that dropouts may not affect these outcomes.

The trial design assumes a 10% dropout rate. However, the toxicity endpoints may be measured very early in the trial, if assuming that dropouts do not affect these endpoints. Practically thus, for every individual a drop-out time was simulated from an exponential distribution with a rate leading to a probability of 10% for dropping out within 2 years. All patients with a simulated dropout time before 2 years, and with no event on EFS, have their EFS value replaced by a missing value in the simulations. For patients with a dropout time before 2 years and with an event on EFS, the procedure is slightly different. The reason is that the dropout time of these patients may happen after experiencing the event on EFS. For these patients, no missing value is thus to be considered for the binary EFS. In practice, for all patients having a combination of an EFS event and a dropout time before 2 years, a time to event for EFS was simulated. Denoting this time by T, realizations of T was simulated by drawing from a conditional exponential distribution F_{T|T}<₂, where F_{T} is an Exponential distribution with a rate such that the assumed probability of an event on EFS within 2 years (Table 1 of the paper). Once the time to event on EFS is simulated, the latter was compared with the time to dropout. If the time to dropout occurs before the time to event on EFS, the value of the endpoint is replaced by a missing value.

Once the procedure to simulate dropouts in the trial was established, one needs to account for these in Generalized Pairwise Comparisons. A naive way of handling missingness in GPC may be to consider as neutral for the endpoint under evaluation all pairs that are composed of at least one missing value. For instance, if a pair on EFS is composed of (NA, 0), then the pair may be classified on lower priority endpoints. In the current design however, this procedure is inappropriate. The reason for this, is that missingness only affects EFS. As a result, this naive handling of missing data reduces the weight of EFS on the definition of the NTB, in favor of the toxicity endpoints. As the latter are expected to favor the treatment arm, reducing the weight of EFS may result in a higher NTB than in the case of no missingness, which, in turn, may lead to a smaller required sample size for the trial.

As the above-described procedure for handling missingness may inappropriately reduce the sample size required, it is preferred to impute the missing observations based on the available information. That is, when simulating a trial with missing data, one first needs to consider a technique to impute the missing observations on EFS based on the other endpoints, and then analyze this completed dataset with GPC. For each simulated dataset, the procedure of imputing and analyzing is repeated several times (10 times), and the 10 different estimated variances for the test statistic relative to the NTB are then aggregated in order to obtain a single p-value, as may have been the case if no imputation was needed. The aggregation of variances follows the approach of Rubin (1987).¹ In particular, letting \hat{Δ}ⱼ denote the estimated NTB for the j-th imputed dataset (j=1, . . . , 10), the ‘aggregated’ two-sided p-value is obtained by computing

$$2 * {P}_{{H}_{0}} ( T > \vert {T}_{o b s} \vert ) ,$$

where T follows a Student distribution with degrees of freedom described hereunder, T_{obs} is the observed value for the aggregated test statistic computed as

$$\overline{\hat{Δ}} / \sqrt{V} for \overline{\hat{Δ}} = 1 / 10 * {\sum }_{j = 1}^{10} {\hat{Δ}}_{j} , and V = W + ( 1 + \frac{1}{10} ) * B , with B = 1 / 9 * {\sum }_{j = 1}^{10} {( {\hat{Δ}}_{j} - \overline{\hat{Δ}} )}^{2} and W = 1 / 10 * {\sum }_{j = 1}^{10} {\hat{V}}_{j}$$

with \hat{V}ⱼ the estimated variance of the test statistic for the j-th imputed dataset. The degrees of freedom are selected here with the particular case of 10 imputations as 4+5*(1+7/(9*r))², with

$$r = ( 1 + \frac{1}{10} ) * B / W$$

(see e.g. Schafer (1997)).²

In order to apply this iterative procedure, a method for imputing values at each iteration was required. For this task, the procedure described for instance in White et al. (2011) was utilized. (White I R, Royston P, Wood A M. Multiple imputation using chained equations: Issues and guidance for practice. Stat Med 2011; 30: 377-99. DOI: 10.1002/sim.4607, which publication is hereby incorporated by reference in its entirety for all purposes). In particular, as data are simulated with a given dependence structure, the observed data for the toxicity endpoints was used in order to impute missing EFS values. In practice, for each simulated trial, a logistic regression model was used per treatment arm for the EFS indicator with the four remaining endpoints as covariates. This leads to an estimated vector parameter \hat{β} along with its estimated variance matrix V. For each of the 10 imputations, a new vector β* from a MVN(\hat{β}, V) was drawn. For each missing value on EFS, let

$${p}_{i}^{*} = {( 1 + exp ( - {β}^{*} {x}_{i} ) )}^{- 1}$$

be the probability of an event on EFS for an individual with toxicity values xi. An imputed value for EFS is then obtained from a Bernoulli sampling with this probability p*ᵢ. This two-step sampling process is then repeated 10 times for each simulated trial.
7. Thresholds of clinical relevance. For outcomes that are ordered (including continuous outcomes), a threshold of clinical relevance can be specified so that a difference smaller than the threshold is ignored in pairwise comparisons. For example, a difference of less than 3 months in survival might be considered too small to be of clinical relevance. In this case the definition of the pairwise score is defined as follows for the i^{th} patient in the treatment group and the j^{th} patient in the control group:

$${u}_{i j} = \{ \begin{matrix}+ 1 if {X}_{i} - {Y}_{j} > τ \\ - 1 if {X}_{i} - {Y}_{j} < - τ \\ 0 otherwise\end{matrix}$$

where “τ” denotes the threshold of clinical relevance.
7. The Net Treatment Benefit. The Net Treatment Benefit is estimated as the mean pairwise score over all the pairs that can be formed between one patient from the treatment group and one patient from the control group:

$$\hat{Δ} = {\sum }_{i = 1}^{{n}_{T}} {\sum }_{j = 1}^{{n}_{C}} {u}_{i j} / {n}_{T} \cdot {n}_{C} ,$$

where \hat{Δ} denotes the estimated Net Treatment Benefit, n_{T} denotes the number of patients in the treatment group and n_{C} the number of patients in the control group. For a single outcome, the Net Treatment Benefit Δ represents the probability that a random subject in the treatment group does better than a random subject in the control group, minus the probability of the opposite situation. For a single outcome with a threshold of clinical relevance, the Net Treatment Benefit Δ represents the probability that a random subject in the treatment group does better by at least the threshold of clinical relevance than a random subject in the control group, minus the probability of the opposite situation (Peron J, Roy P, Ozenne B, et al. The net chance of a longer survival as a patient-oriented measure of treatment benefit in randomized clinical trials. JAMA Oncol 2016; 2: 901-905, which publication is hereby incorporated by reference in its entirety for all purposes.) For multiple prioritized outcomes, the Net Treatment Benefit Δ represents the probability that a random subject in the treatment group does better than a random subject in the control group, either for the outcome of highest priority, or, in case of a tie for the outcome of highest priority, for the outcome of next highest priority, and so on, minus the probability of the opposite situation.
8. Benefit/risk analyses. Generalized Pairwise Comparisons of prioritized outcomes allows arbitrarily complex benefit/risk analyses to be conducted in a mathematically rigorous way. In contrast to marginal analyses of each outcome taken separately, it allows the analyst to estimate a Net Treatment Benefit that accounts for the correlation between the various outcomes to be taken into account (Buyse et al., 2021. The net benefit of a treatment should take the correlation between benefits and harms into account. J Clin Epidemiol 2021; 137: 148-158, which publication is hereby incorporated by reference in its entirety for all purposes.) This approach has been used to investigate the benefit/risk of three experimental treatments compared with gemcitabine for advanced pancreatic cancer: erlotinib, FOLFORINOX and gemcitabine+nab-paclitaxel (Péron et al. The net chance of a longer survival as a patient-oriented measure of treatment benefit in randomized clinical trials. JAMA Oncol 2016; 2: 901-905, which publication is hereby incorporated by reference in its entirety for all purposes.)

### Results

### 1. Sample Size Calculations

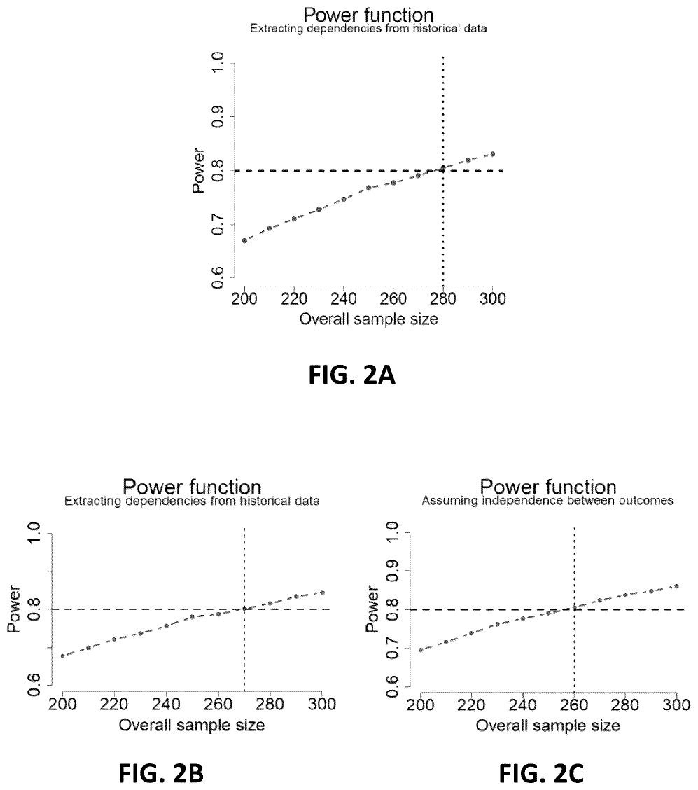

When considering the dependence structure extracted from historical data, simulation results led to a total sample size of 280 patients (140 per group) for an approximate power of 80% (FIG. 2A).

When repeating the simulations assuming no dropouts and complete independence between the outcomes, slightly different sample sizes were obtained, as shown in FIG. 2B and FIG. 2C respectively. The dependence structure based on historical data leads to a slightly larger sample size (N=280, FIG. 2A) than when assuming complete independence (N=260, FIG. 2C). The no-dropout situation also leads to a smaller required sample size (N=270, FIG. 2B), although the decrease in sample size is attenuated by the multiple imputation. In contrast, if the missingness in the data had been handled by disregarding all patients with a missing value on EFS, the sample size may have grown to ˜300 (=270/(1−10%)).

### 2. NTB

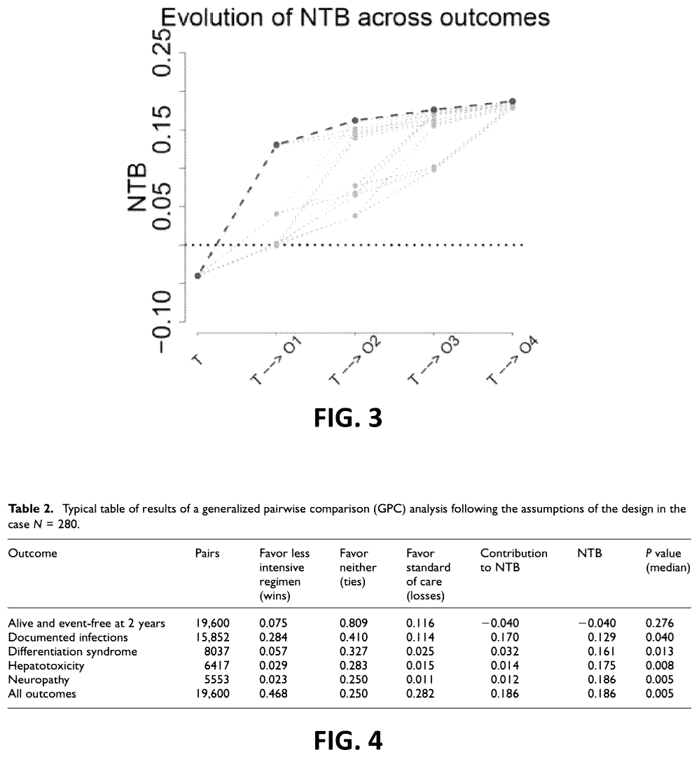

Across all simulations, the NTB expected in the present trial was equal to 0.19 (range −0.06 to 0.40, and interquartile range 0.14-0.23 for sample size N=280). The NTB is interpreted as a difference between the probability of doing better on the less intensive treatment regimen than on the standard of care, minus the probability of the opposite situation. A NTB of 0.19 translates to a number needed to treat of about 5.3 patients (1/0.19˜5.3). FIG. 3 shows the evolution of the cumulative NTB value across all prioritized outcomes in an example simulation. The first bullet represents the NTB for EFS only. The observed value is below 0, which reflects the small deterioration expected (24%, as shown in Table 1 (FIG. 1), which is exactly equal to the NTB). The second bullet represents the value of the NTB only accounting for the first two outcomes in calculating the NTB, namely EFS and documented infections. The graph thus shows the evolutive nature of the NTB as more and more outcomes are taken into account and illustrates that a small deterioration in terms of EFS is overcome by the multiple gains on toxicity outcomes. The other curves in FIG. 3 show for their part the evolution of different NTBs when one shuffles the order of priorities across outcomes.

In particular, while EFS is always taken as first priority, all possible permutations of the ordering for the toxicity outcomes are then considered in the graph. The second bullets starting from EFS thus show four potential scenarios, for the four choices of toxicity that may be considered as second priority in the NTB. The abscissa axis refers to the outcomes composing the NTB, with these being potentially different depending on the order of priorities. In total, shuffling the order of priorities leads to adding 23 extra curves to the graph (not all being distinct as some over-lap exists), depicted in gray. The point of interest is to note that, although trajectories vary across outcomes, all potential orderings of the toxicity outcomes lead to an overall NTB that is of the same order of magnitude as the one considered for the design of the trial.

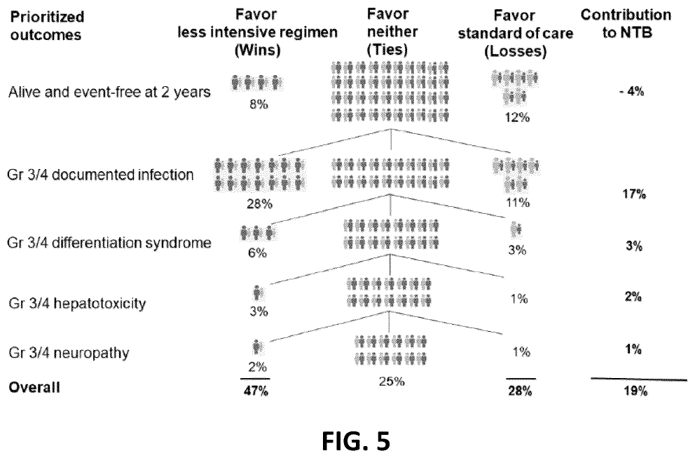

Table 2, as shown in FIG. 4, presents an example of results expected from generalized pairwise comparisons following the assumptions of the design in the situation N=280. This table is obtained by averaging results over the 10,000 simulations, while p-values are reported by taking the median over all simulations. Each line of the table presents the number of pairs evaluated on the outcome of interest, the proportion of pairs favoring the less intensive regimen (favorable), the proportion of pairs favoring neither the less intensive regimen nor the standard of care (Ties), the proportion of pairs favoring the standard of care (unfavorable), the contribution of the outcome to the NTB, and the NTB up to the outcome reported in the line, with a corresponding p-value. FIG. 5 further helps exhibiting this result, by illustrating the classification of pairs graphically at each level of the hierarchical procedure.

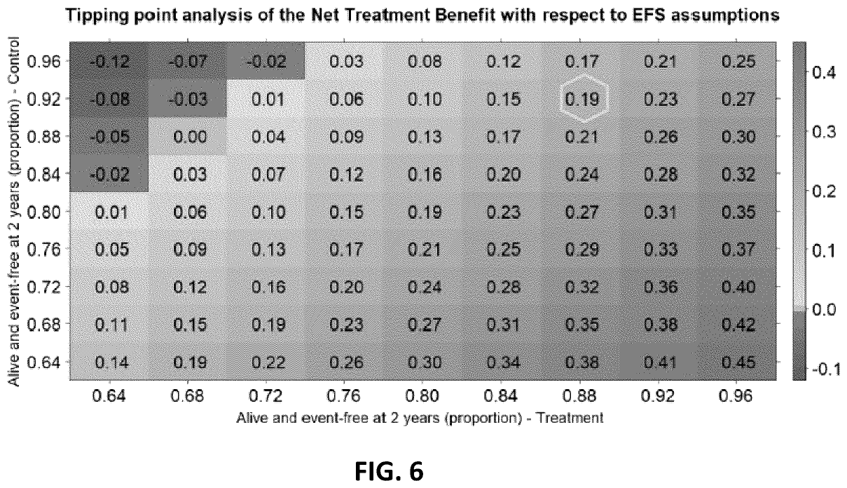

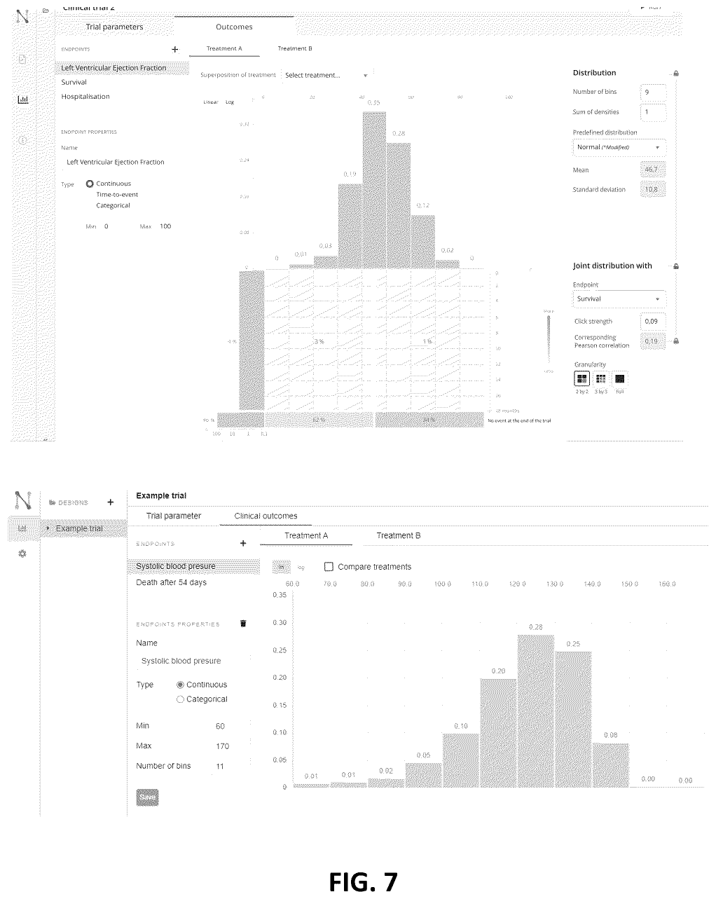

FIG. 6 depicts a tipping-point analysis for the assumptions on EFS. That is, with the same order of priorities as in our design, the assumptions on EFS in each treatment group were allowed to vary and the NTBs for each set of assumptions was calculated to assess the sensitivity of the balance between efficacy and toxicities in this context. FIG. 6 shows that, keeping the toxicity and dependencies assumptions fixed, the NTBs may remain positive for a wide range of assumptions on EFS in both groups. And as expected, in scenarios where EFS assumptions are the same in both arms, the NTBs may have decreasing values with higher proportions of EFS events (e.g., on the main diagonal). This is because higher EFS proportions (up to 50%) may result in smaller proportions of pairs declared neutral on EFS, hence decreasing the (positive) contribution to the NTBs of the toxicity outcomes.

### Discussion

Provided herein is an innovative approach to test a reduction in dose and schedule intensity using an example in patients with APL treated with ATRA. The approach is original because it combines multiple prioritized outcomes in a single analysis, with the efficacy outcomes (here, EFS at 2 years) having higher priority, and tolerability outcomes having lower priorities (here, four pre-specified toxicities known to occur with high frequency in this clinical situation). A clinically sensible order of priorities was used to calculate the expected NTB through simulations, and these simulations were used to calculate the sample size that may provide 80% power for postulated effects of a less intensive treatment regimen on EFS and the toxicities of interest. Of note, the order in which toxicities are prioritized may be changed according to individual preferences on the part of patient or other stakeholders, such as trialists and regulators. The impact of changing the order of priorities was gauged by calculating the distribution of the NTB across all orders of priority. It was concluded that a sample size of about 280 patients may be sufficient to establish the superiority of a less intensive treatment regimen after penalizing this experimental treatment for its potentially lower efficacy.

To be clear, the approaches provided herein may not replace NI trials. In situations where NI must be established beyond a reasonable doubt, there may be no alternative to conducting an NI trial with a prespecified, acceptable NI margin. In such trials, for a time to event, such as EFS, the upper limit of the hazard ratio's confidence interval may have to be below the NI margin. Although this approach is theoretically feasible in all cases, the sample size required for a well-powered NI trial may be prohibitive. In our example, if a one-sided significance level of 5% was used, and 80% power was required to establish the NI of the less intensive regimen, with a margin of 4% (absolute difference in EFS rates at 2 years), assuming the two treatments have an identical 2-year EFS rate of 92%, at least 700 patients may be required, and 140 events observed after 7 years of follow-up. Note that the margin of 4% as an absolute difference in 2-year EFS translates to a hazard ratio of about 1.5, which is already a large margin as it means a 50% increase in the risk for the patients to have a disease recurrence or to die within the first 2 years. Such an NI trial is clearly much more challenging to conduct, especially for a relatively rare disease, such as APL. If the NI trial was attempted anyway, there may be a high risk of early termination due to lack of sufficient accrual, or of the question becoming obsolete before the trial is completed.

In the approach adopted here, EFS is analyzed as the first outcome, and a test of hypothesis can be conducted on this outcome alone if desired, but that may not be the goal pursued. Instead, presented herein is a situation in which clinical investigators may consider the point estimate of the EFS difference and its 95% confidence limits to assess whether the deficit in EFS remains acceptable, given the benefits in toxicity. However, the 95% confidence limits of the EFS difference may be too large to exclude potentially substantial deficits (or benefits) of the experimental treatment. Hence, the approach may be appropriate if such large deficits can be a priori excluded, for instance, if there is pharmacokinetic or pharmacodynamic evidence that the reduced treatment dose is very likely to have similar efficacy.

The approaches provided herein may depend on the duration of follow-up of the study. Indeed, toxicities may occur within a few weeks after starting therapy, whereas the efficacy benefits of treatment occur over a longer period of time. If clinicians wish to focus only on efficacy, the traditional approach of conducting an NI trial using the EFS hazard ratio may remain the gold standard. Under the assumption of proportional hazards, the trial can be analyzed at any time during the follow-up since the hazard ratio is assumed constant over time. In contrast, if clinicians or patients are interested in the trade-off between small deficits in efficacy as com-pared to benefits in tolerability, a time horizon must be specified for the NTB to be estimated. The NTB is an absolute measure of net treatment effect that can complement other commonly used measures, including the hazard ratio. For binary outcomes, such as toxicities, the NTB is equal to the difference in the probability of having the toxicity in the experimental arm and in the control arm. For a time to event, such as EFS, it is the difference in the probability of being alive and event-free in the experimental arm and in the control arm within a pre-specified time horizon. For rare events (say, those that occur in less than 20% of the patients during the follow-up time considered), a test for the difference in probability is almost as powerful as a logrank test that takes the time to these events into account. In the clinical situation considered here, 2 years was deemed an adequate follow-up period, given that recurrences past this point are very rare in APL. Given that less than 10% of patients with APL have a recurrence within 2 years, EFS can be considered a binary outcome without much information, but in general EFS is best analyzed as time to event variable. In some situations, the duration of follow-up may be driven by the time course of the events (for example, in the adjuvant treatment of colorectal cancer, almost all recurrences occur with-in 5 years, whereas in hormone receptor-positive breast cancer, they continue to occur more than a decade after randomization). In other situations, the duration of follow-up may be imposed by practical constraints, such as dropout rates or availability of resources for long-term follow-up. An advantage of the approach provided herein is that all relevant outcomes of any type (time to event, binary, continuous) can be included in a single analysis. This feature of the analysis opens up the possibility of adding patient-reported outcomes in the analysis. It is worth noting that the correlation between the outcomes is automatically taken into account in the analysis, which is desirable to distinguish between toxicities that are correlated with efficacy versus those that are independent of efficacy, and as such less acceptable. The overall NTB can be decomposed into the additive contributions of all outcomes considered, which is extremely useful clinically. In the example discussed here, the NTB for EFS alone may be shown as the first outcome of interest, followed by the additional contributions of the NTB due to toxicities, conditional on efficacy being the same. Such a decomposition of the NTB is potentially quite useful in dialogues about treatment options with patients.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Example 2

This example demonstrates a method for designing a clinical trial evaluating a clinical intervention in trial subjects. The example focuses on the design of a clinical trial with reduced number of necessary subjects, using generalized pairwise comparisons (GPC) to perform a clinical trial for administering a lower dose of a clinical intervention.

Investigators of a clinical trial may need to lower the dose (to reduce toxicity & improve compliance) for the trial without loss of efficacy benefits towards the patients. Using a conventional trial design to prove non-inferiority of the lower dose had a projected sample size of 700 patients. Using the GPC methodology to include both efficacy and safety criteria based on a priority function elicited from the patients and patients representative, to assess the net treatment benefit of the reduced dose, made the trial feasible by reducing the required sample size to only 280 patients.

### Example 3

This example demonstrates how an adaptive, web-based paired-comparison method efficiently captures expert and stakeholder preferences on treatment outcomes, making it highly practical for clinical research. The example illustrates the ability to capture expert or stakeholder preferences.

29 investigators participated voluntarily and anonymously, completing the survey in an average of just nine minutes (range: 4-24 minutes). Investigators selected up to five outcomes from seven candidates; the top five in rank order were overall survival (OS), chosen by 72% of participants, followed by global quality of life (QoL) score, progression-free survival (PFS), side-effect burden, and serious adverse event (SAE) occurrence. The most frequently selected clinically meaningful thresholds were +3 months for OS, +6 months for PFS, and +1 level improvement for QoL and side-effect burden, while SAEs were retained as a binary safety outcome. By systematically formalizing which outcomes and thresholds are most meaningful, this approach ensures that trial endpoints reflect real-world clinical priorities of real-world clinical experts. The process is rapid, transferable across diseases and stakeholder groups, and enables integration of multiple prioritized outcomes into a single Net Treatment Benefit (NTB) measure. Ultimately, this results in a more comprehensive, clinically relevant, and interpretable assessment of treatment effects, aligning trial design and analysis with everyday practice and supporting transparent, patient-centered decision-making.

1.-168. (canceled)

169. A method of using a graphical user interface to assist in determination of a clinical study parameters, comprising:
a) obtaining a dataset for a treatment set of subjects and a reference set of subjects, wherein the treatment set of subjects receives a clinical intervention during the clinical trial and the reference set of subjects does not receive the clinical intervention during the clinical trial, and wherein the dataset comprises a plurality of treatment outcomes for the treatment set of subjects and the reference set of subjects during the clinical trial;
b) receiving, on a first computing device, the plurality of treatment outcomes for the treatment set of subjects and the reference set of subjects, wherein the plurality of treatment outcomes are transmitted over a computer network;
c) presenting to a first user, via a first user interface on a first electronic display of the first computing device, a representation of the plurality of treatment outcomes;
d) selecting, by the first user via the first user interface on the electronic display, a prioritization function that assigns ranked values to each of the plurality of treatment outcomes, wherein the ranked values are selected by the first user based at least in part on (1) subject-level efficacies and subject-level adverse effects of individual treatment outcomes of the plurality of treatment outcomes on the subject and (2) a personalized preference of the subject;
e) performing, by a computer processor, a plurality of simulated clinical trials using the dataset and the prioritization function, thereby producing a set of simulated outcomes;
f) processing the set of simulated outcomes to generate a set of simulated net treatment benefit scores and a set of simulated trial power scores, wherein the processing comprises performing a set of pairwise comparisons between a first subject selected from the treatment set of subjects and a second subject selected from the reference set of subjects for each of the simulated outcomes in the set of simulated outcomes;
g) determining a parameter for the clinical trial based on the set of simulated net treatment benefit scores and the set of simulated trial power scores; and
h) presenting to a second user, via a second user interface on a second electronic display of a second computing device, a report comprising the parameter for the clinical trial.

170. The method of claim 169, wherein the parameter of the clinical trial comprises power, alpha, a sample size, an endpoint of the trial, or any combination thereof.

171. The method of claim 170, wherein the sample size is at most about 500, at most about 400, at most about 300, at most about 200, at most about 100, or at most about 50 subjects.

172. The method of claim 170, wherein the endpoint of the trial comprises a plurality of ranked or prioritized endpoints.

173. The method of claim 169, further comprising modeling a set of variables of the plurality of simulated clinical trials.

174. The method of claim 173, further comprising modeling the set of variables of the plurality of simulated clinical trials based on historical data.

175. The method of claim 173, wherein the set of variables comprises a dropout rate of the plurality of simulated clinical trials, endpoint, a priority of the plurality of treatment outcomes, a structure of dependency of the plurality of treatment outcomes, a variance, a threshold of clinical relevance, or any combination thereof.

176. The method of claim 175, wherein d) further comprises receiving a user input through the computing device.

177. The method of claim 176, wherein d) further comprises receiving the user input, thereby generating the prioritization function at least in part based on the user input.

178. The method of claim 176, wherein d) further comprises receiving the user input, thereby determining the dependency of the plurality of treatment outcomes at least in part based on the user input.

179. The method of claim 176, wherein d) further comprises receiving the user input, thereby determining the threshold of clinical relevance at least in part based on the user input.

180. The method of claim 176, wherein d) further comprises receiving the user input on a set of pairwise clinical scenarios.

181. The method of claim 180, wherein the user input represents a candidate subject of the clinical trial.

182. The method of claim 180, where the user input represents a subject of the clinical trial.

183. The method of claim 180, wherein the set of pairwise clinical scenarios comprise at least 10, at least 50, or at least 100 sets of pairwise clinical scenarios.

184. The method of claim 183, wherein the pairwise clinical scenarios comprise administration of the clinical intervention in the subject.

185. The method of claim 184, wherein the clinical intervention comprises an experimental drug treatment.

186. The method of claim 169, wherein the first computing device and the second computing device are the same, and wherein the first user and the second user are the same.

187. The method of claim 169, wherein the plurality of treatment outcomes comprises a member selected from the group consisting of event-free survival time, progression-free survival time, overall survival time, another time to event, efficacy, safety, quality of life, an adverse event, a score, and a biomarker.

188. The method of claim 169, wherein the set of simulated net treatment benefit scores comprises a member selected from the group consisting of event-free survival time, progression-free survival time, overall survival time, another time to event, efficacy, safety, quality of life, an adverse event, a score, a biomarker, a reaction, a side effect, and a toxicity of the clinical intervention.
